(19) United States
(12) Patent Application Publication
(10) Pub. No.: US 2025/0042894 A1
(43) Pub. Date: Feb. 6, 2025

(54) PROTEIN TYROSINE PHOSPHATASE INHIBITORS AND METHODS OF USE THEREOF

(71) Applicants: Calico Life Sciences LLC, South San Francisco, CA (US); AbbVie Inc., North Chicago, IL (US)

(72) Inventors: Andrew Bogdan, Evanston, IL (US); Christos Economou, Wilmette, IL (US); Jennifer M. Frost, Gurnee, IL (US); Philip R. Kym, Libertyville, IL (US); Spencer O. Scholz, San Francisco, CA (US); Zhaoming Xiong, Cambridge, MA (US)

(21) Appl. No.: 18/709,204

(22) PCT Filed: Nov. 10, 2022
(86) PCT No.: PCT/US2022/049581

§ 371 Date: May 10, 2024

Related U.S. Application Data
(60) Provisional application No. 63/278,336, filed on Nov. 11, 2021.

(51) Int. Cl. C07D 417/14; A61K 31/433; C07D 417/04

(52) U.S. Cl. CPC C07D 417/14; A61K 31/433; C07D 417/04

(57) ABSTRACT

The present invention provides for compounds of Formula (I) wherein R¹, has any of the values defined herein, and pharmaceutically acceptable salts thereof, that are useful as agents in the treatment of non-small cell lung cancer.

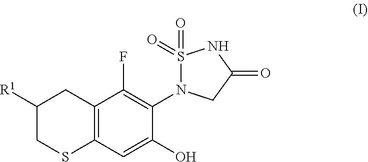

### CROSS-REFERENCE TO RELATED APPLICATION

This application claims the benefit of and priority to U.S. Provisional Application No. 63/278,336, filed on Nov. 11, 2021, the entire disclosure of which is hereby incorporated by reference herein in its entirety for all purposes.

### BACKGROUND

Protein tyrosine phosphatase non-receptor type 2 (PTPN2), also known as T cell protein tyrosine phosphatase (TC-PTP), controls multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA 89:499-503; 1992). In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif (Tillmann U. et al., Mol Cell Biol 14:3030-3040; 1994). The 45 kDa isoform can passively transfuse into the cytosol under certain cellular stress conditions. PTPN2 negatively regulates signaling of non-receptor tyrosine kinases (e.g. JAK1, JAK3), receptor tyrosine kinases (e.g. INSR, EGFR, CSF1R, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ.

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T. et al., Nature 547:413-418; 2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways (Kenner K. A. et al., J Biol Chem 271: 19810-19816, 1996). Animals deficient in PTPN1 have improved glucose regulation and lipid profiles and are resistant to weight gain when treated with a high fat diet (Elchebly M. et al., Science 283: 1544-1548, 1999). Thus, there is a medical need for PTPN1 inhibitors.

### BRIEF SUMMARY

The present disclosure is directed, at least in part, to compounds, for the inhibition of protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTP1B)).

For example, disclosed herein is a compound represented by Formula (I):

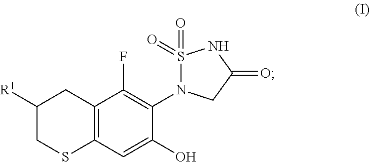

or a pharmaceutically acceptable salt thereof, wherein:
R¹ is selected from the group consisting of —NH₂, —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl;
   wherein —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
   wherein if —N(R)—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}; and
R^{g} is independently selected, for each occurrence, from the group consisting of halogen, hydroxyl, C₁₋₆alkyl, phenyl, and C₁₋₆alkoxy, wherein C₁₋₆alkyl, phenyl, or C₁₋₆alkoxy may optionally be substituted by one, two three or more substituents each independently selected from R^{P};
R^{h} is independently selected, for each occurrence, from the group consisting of C₁₋₆alkyl and C₁₋₆alkyl-O—C(O)—;
R^{P} is independently selected, for each occurrence, from the group consisting of C₁₋₆alkyl, halogen and hydroxyl; and
R^{a} is independently selected, for each occurrence, from the group consisting of hydrogen and C₁₋₆alkyl.

### BRIEF DESCRIPTION OF THE SEQUENCE LISTING

Incorporated herein by reference in its entirety is a Sequence Listing entitled, CLS-028PR_ABV21372USL1 SEQ ID List_ST25.txt”, comprising SEQ ID NO: 1 through SEQ ID NO: 3, which includes the amino acid sequence disclosed herein. The Sequence Listing has been submitted herewith in ASCII text format via EFS. The Sequence Listing was first created on Jun. 22, 2021 and is 7,282 bytes in size.

### DETAILED DESCRIPTION

For example, disclosed herein is a compound represented by Formula (I):

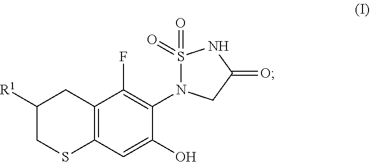

or a pharmaceutically acceptable salt thereof, wherein:
R¹ is selected from the group consisting of —NH₂, —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl;
   wherein —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
   wherein if —N(R)—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}; and
R^{g} is independently selected, for each occurrence, from the group consisting of halogen, hydroxyl, C₁₋₆alkyl, phenyl, and C₁₋₆alkoxy, wherein C₁₋₆alkyl, phenyl, or C₁₋₆alkoxy may optionally be substituted by one, two three or more substituents each independently selected from R^{P};
R^{h} is independently selected, for each occurrence, from the group consisting of C₁₋₆alkyl and C₁₋₆alkyl-O—C(O)—;
R^{P} is independently selected, for each occurrence, from the group consisting of C₁₋₆alkyl, halogen and hydroxyl; and
R^{a} is independently selected, for each occurrence, from the group consisting of hydrogen and C₁₋₆alkyl.

In some embodiments, R¹ is selected from the group consisting of —NH₂, —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl;
wherein —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
wherein if —N(R)—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}

In some embodiments, R¹ is —N(H)—C₁₋₈alkyl, wherein C₁₋₈alkyl is optionally substituted with R^{g}. In some embodiments, R¹ is —N(H)—C₁₋₈alkyl. In some embodiments, R¹ is —N(H)—C₁₋₆alkyl, wherein C₁₋₆alkyl is optionally substituted with R^{g}. In some embodiments, R¹ is —N(H)—C₁₋₈alkyl, wherein C₁₋₈alkyl is optionally substituted with R^{g}, wherein R^{g} is selected from fluoro, hydroxyl, C₁₋₆alkyl, and C₁₋₆alkoxy, wherein C₁₋₆alkyl, and C₁₋₆alkoxy are optionally substituted by one, two three or more substituents selected from R; and R is independently selected, for each occurrence, from halogen. In some embodiments, R¹ is —N(H)—C₁₋₈alkyl, wherein C₁₋₈alkyl is substituted with one or two instances of halogen, C₁₋₆alkyl, or C₁₋₆alkoxy. In some embodiments, R¹ is selected from the group consisting of

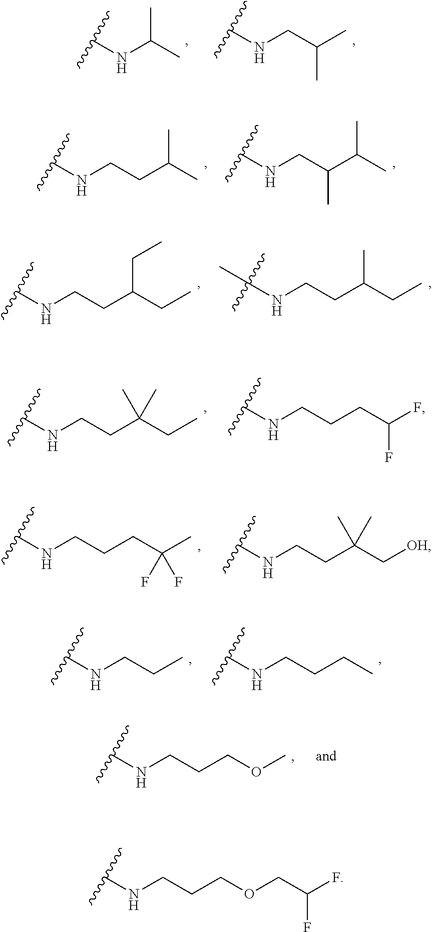

In some embodiments, R¹ is —N(H)—C₁₋₆alkylene-C₃₋₆cycloalkyl, wherein R¹ may optionally be substituted by one, two, three or more substituents each independently selected from R^{g}. In some embodiments, R¹ is —N(H)—C₁₋₆alkylene-C₃₋₆cycloalkyl may optionally be substituted by one, two, three or more substituents, R^{g}; wherein R^{g} is independently selected, for each occurrence, from the group consisting of fluoro, C₁₋₆alkyl, and phenyl, wherein C₁₋₆alkyl and phenyl are optionally substituted by one, two three or more substituents selected from R^{P}; and R^{P} is independently selected, for each occurrence, from fluoro or hydroxyl. In embodiments, is —N(H)—C₁₋₆alkylene-C₃₋₆cycloalkyl, wherein R¹ may optionally be substituted by one, two, three or more substituents each independently selected from the group consisting of fluorine, C₁₋₆alkyl, and phenyl, wherein C₁₋₆alkyl may optionally be substituted by one, two or three fluorine. In embodiments, is —N(H)—C₁₋₆alkylene-C₃₋₆cycloalkyl, wherein R¹ is substituted with one, two, three or more substituents each independently selected from the group consisting of fluorine, C₁₋₆alkyl, and phenyl, wherein C₁₋₆alkyl may optionally be substituted by one, two or three fluorine.

In some embodiments, R¹ is selected from the group consisting of

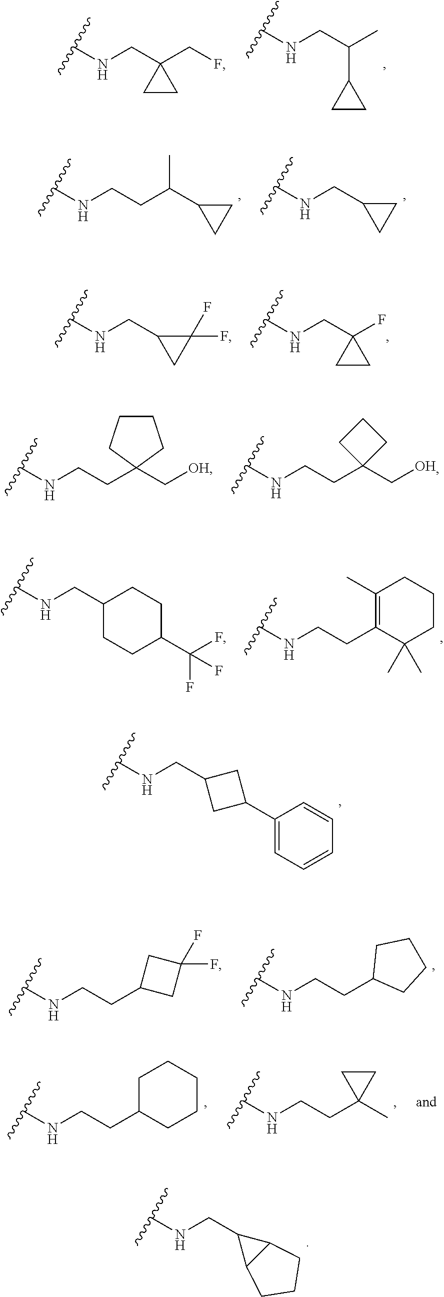

In some embodiments, R¹ is —N(H)—C₁₋₆alkylene-4-6 membered heterocyclyl, wherein R¹ may optionally be substituted by one, two, three or more substituents each independently selected from R^{g}, wherein if 4-7 membered heterocyclyl contains a substitutable ring nitrogen atom, that ring nitrogen atom may optionally be substituted by R^{h}. In some embodiments, R¹ is —N(H)—C₁₋₆alkylene-4-6 membered heterocyclyl, wherein R¹ is substituted with one, two, three or more substituents each independently selected from R^{g}, wherein if 4-7 membered heterocyclyl contains a substitutable ring nitrogen atom, that ring nitrogen atom may optionally be substituted by R^{h}. In some embodiments, R¹ is —N(H)—C₁₋₆alkylene-4-6 membered heterocyclyl, wherein R¹ is substituted with one, two, three or more substituents each independently selected from R^{g}, wherein if 4-7 membered heterocyclyl contains a substitutable ring nitrogen atom, that ring nitrogen atom may optionally be substituted by R^{h}, and R^{h} is C₁₋₆alkyl-O—C(O)—. In some embodiments, R¹ is —N(H)—C₁₋₆alkylene-4-6 membered heterocyclyl, wherein R¹ may optionally be substituted by one, two, three or more substituents each independently selected from the group consisting of fluorine, C₁₋₆alkyl, and phenyl, wherein C₁₋₆alkyl may optionally be substituted by one, two or three fluorine.

In some embodiments, R¹ is selected from the group consisting of

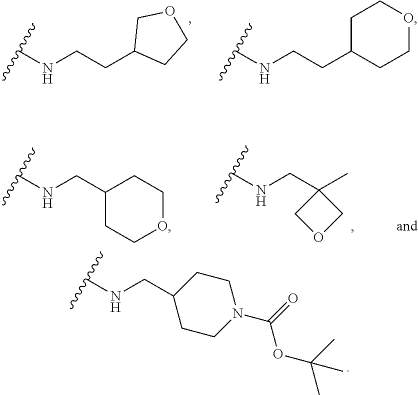

In some embodiments, R¹ is —N(H)—C₁₋₆alkylene-phenyl, wherein R¹ may optionally be substituted on one or more available carbons by one, two, three or more substituents each independently selected from R^{g}. In some embodiments, R¹ is —N(H)—C₁₋₆alkylene-phenyl, wherein R¹ may optionally be substituted on one or more available carbons by one, two, three or more substituents each independently selected from R^{g}, and R^{g} is independently, for each occurrence, C₁₋₆alkyl.

In some embodiments, R¹ is selected from the group consisting of

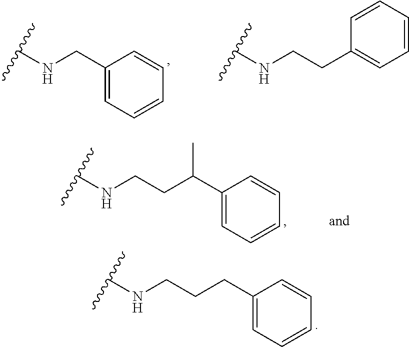

In some embodiments, R¹ is —N(H)—C₁₋₆alkylene-5-6 membered heteroaryl, wherein R¹ may optionally be substituted on one or more available carbons by one, two, three or more substituents each independently selected from R^{g}. In some embodiments, R¹ is —N(H)—C₁₋₆alkylene-5-6 membered heteroaryl, wherein R¹ may optionally be substituted on one or more available carbons by one, two, three or more substituents each independently selected from R^{g}, and R^{g} is independently, for each occurrence, C₁₋₆alkyl.

In some embodiments, R¹ is

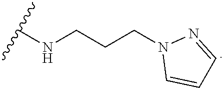 [SMILES: *NCCCn1cccn1]

The present disclosure includes a compound selected from the group consisting of
5-{(3S)-3-[(4,4-difluorobutyl)amino]-5-fluoro-7-hydroxy-3,4-dihydro-2H-1-benzothiopyran-6-yl}-1λ⁶,2,5-thiadiazolidine-1,1,3-trione;
tert-butyl [(3S)-5-fluoro-7-hydroxy-6-(1,1,4-trioxo-1λ⁶,2,5-thiadiazolidin-2-yl)-3,4-dihydro-2H-1-benzothiopyran-3-yl]carbamate;
5-[(3S)-3-amino-5-fluoro-7-hydroxy-3,4-dihydro-2H-1-benzothiopyran-6-yl]-1λ⁶,2,5-thiadiazolidine-1,1,3-trione;
5-{(3S)-5-fluoro-7-hydroxy-3-[(3-methylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-({2-[1-(hydroxymethyl)cyclobutyl]ethyl}amino)-3,4-dihydro-2H-1-benzothiopyran-6-yl]-1λ⁶,2,5-thiadiazolidine-1,1,3-trione;
5-[(3S)-5-fluoro-7-hydroxy-3-({2-[1-(hydroxymethyl)cyclopentyl]ethyl}amino)-3,4-dihydro-2H-1-benzothiopyran-6-yl]-1λ⁶,2,5-thiadiazolidine-1,1,3-trione;
5-{(3S)-5-fluoro-7-hydroxy-3-[(4-hydroxy-3,3-dimethylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-(propylamino)-3,4-dihydro-2H-1-benzothiopyran-6-yl]-1λ⁶,2,5-thiadiazolidine-1,1,3-trione;
5-{(3S)-5-fluoro-7-hydroxy-3-[(3-methylpentyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[2-(oxan-4-yl)ethyl]amino}-3,4-dihydro-2H-1-benzothiopyran-6-yl]-1λ⁶,2,5-thiadiazolidine-1,1,3-trione;
5-[(3S)-5-fluoro-7-hydroxy-3-{[(oxan-4-yl)methyl]amino}-3,4-dihydro-2H-1-benzothiopyran-6-yl]-1λ⁶,2,5-thiadiazolidine-1,1,3-trione;
5-{(3S)-3-[(cyclopropylmethyl)amino]-5-fluoro-7-hydroxy-3,4-dihydro-2H-1-benzothiopyran-6-yl}-1λ⁶,2,5-thiadiazolidine-1,1,3-trione;
5-[(3S)-3-(butylamino)-5-fluoro-7-hydroxy-3,4-dihydro-2H-1-benzothiopyran-6-yl]-1λ⁶,2,5-thiadiazolidine-1,1,3-trione;
5-{(3S)-5-fluoro-7-hydroxy-3-[(2-methylpropyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[2-(oxolan-3-yl)ethyl]amino}-3,4-dihydro-2H-1-benzothiopyran-6-yl]-1,2,5-thiadiazolidine-1,1,3-trione;
5-{(3S)-3-[(2,3-dimethylbutyl)amino]-5-fluoro-7-hydroxy-3,4-dihydro-2H-1-benzothiopyran-6-yl}-1λ⁶,2,5-thiadiazolidine-1,1,3-trione;
5-{(3S)-5-fluoro-7-hydroxy-3-[(propan-2-yl)amino]-3,4-dihydro-2H-1-benzothiopyran-6-yl}-1λ⁶,2,5-thiadiazolidine-1,1,3-trione;
5-[(3S)-3-{[2-(3,3-difluorocyclobutyl)ethyl]amino}-5-fluoro-7-hydroxy-3,4-dihydro-2H-1-benzothiopyran-6-yl]-1λ⁶,2,5-thiadiazolidine-1,1,3-trione;
5-{(3S)-3-[(2-cyclohexylethyl)amino]-5-fluoro-7-hydroxy-3,4-dihydro-2H-1-benzothiopyran-6-yl}-1λ⁶,2,5-thiadiazolidine-1,1,3-trione;
5-{(3S)-3-[(3-ethylpentyl)amino]-5-fluoro-7-hydroxy-3,4-dihydro-2H-1-benzothiopyran-6-yl}-1λ⁶,2,5-thiadiazolidine-1,1,3-trione;
5-{(3S)-3-[(2-cyclopentylethyl)amino]-5-fluoro-7-hydroxy-3,4-dihydro-2H-1-benzothiopyran-6-yl}-1λ⁶,2,5-thiadiazolidine-1,1,3-trione;
5-[(3S)-3-(benzylamino)-5-fluoro-7-hydroxy-3,4-dihydro-2H-1-benzothiopyran-6-yl]-1λ⁶,2,5-thiadiazolidine-1,1,3-trione;
5-{(3S)-5-fluoro-7-hydroxy-3-[(3-methylbut-2-en-1-yl)amino]-3,4-dihydro-2H-1-benzothiopyran-6-yl}-1λ⁶,2,5-thiadiazolidine-1,1,3-trione;
5-{(3S)-5-fluoro-7-hydroxy-3-[(2-phenylethyl)amino]-3,4-dihydro-2H-1-benzothiopyran-6-yl}-1λ⁶,2,5-thiadiazolidine-1,1,3-trione;
5-{(3S)-3-[(3,3-dimethylpentyl)amino]-5-fluoro-7-hydroxy-3,4-dihydro-2H-1-benzothiopyran-6-yl}-1λ⁶,2,5-thiadiazolidine-1,1,3-trione;
5-[(3S)-5-fluoro-3-1{[(1-fluorocyclopropyl)methyl]amino}-7-hydroxy-3,4-dihydro-2H-1-benzothiopyran-6-yl]-1λ⁶,2,5-thiadiazolidine-1,1,3-trione;
5-[(3S)-3-[{[(2,2-difluorocyclopropyl)methyl]amino}-5-fluoro-7-hydroxy-3,4-dihydro-2H-1-benzothiopyran-6-yl]-1λ⁶,2,5-thiadiazolidine-1,1,3-trione;
5-[(3S)-5-fluoro-7-hydroxy-3-{[(3-methyloxetan-3-yl)methyl]amino}-3,4-dihydro-2H-1-benzothiopyran-6-yl]-1λ⁶,2,5-thiadiazolidine-1,1,3-trione;
5-[(3S)-5-fluoro-3-({[1-(fluoromethyl)cyclopropyl]methyl}amino)-7-hydroxy-3,4-dihydro-2H-1-benzothiopyran-6-yl]-1λ⁶,2,5-thiadiazolidine-1,1,3-trione;
5-{(3S)-3-[(4,4-difluoropentyl)amino]-5-fluoro-7-hydroxy-3,4-dihydro-2H-1-benzothiopyran-6-yl}-1λ⁶,2,5-thiadiazolidine-1,1,3-trione;
5-{(3S)-5-fluoro-7-hydroxy-3-[(3-methoxypropyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[3-(1H-pyrazol-1-yl)propyl]amino}-3,4-dihydro-2H-1-benzothiopyran-6-yl]-1λ⁶,2,5-thiadiazolidine-1,1,3-trione;
5-[(3S)-5-fluoro-7-hydroxy-3-{[2-(1-methylcyclopropyl)ethyl]amino}-3,4-dihydro-2H-1-benzothiopyran-6-yl]-1λ⁶,2,5-thiadiazolidine-1,1,3-trione;
5-{(3S)-3-[(2-cyclopropylpropyl)amino]-5-fluoro-7-hydroxy-3,4-dihydro-2H-1-benzothiopyran-6-yl}-1,2,5-thiadiazolidine-1,1,3-trione;
5-{(3S)-5-fluoro-7-hydroxy-3-[(3-phenylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[(3-phenylcyclobutyl)methyl]amino}-3,4-dihydro-2H-1-benzothiopyran-6-yl]-1λ⁶,2,5-thiadiazolidine-1,1,3-trione;
5-{(3S)-5-fluoro-7-hydroxy-3-[(3-phenylpropyl)amino]-3,4-dihydro-2H-1-benzothiopyran-6-yl}-1λ⁶,2,5-thiadiazolidine-1,1,3-trione;
5-[(3S)-3-{[3-(2,2-difluoroethoxy)propyl]amino}-5-fluoro-7-hydroxy-3,4-dihydro-2H-1-benzothiopyran-6-yl]-1λ⁶,2,5-thiadiazolidine-1,1,3-trione;
5-[(3S)-3-({[(1RS,5SR)-bicyclo[3.1.0]hexan-6-yl]methyl}amino)-5-fluoro-7-hydroxy-3,4-dihydro-2H-1-benzothiopyran-6-yl]-1λ⁶,2,5-thiadiazolidine-1,1,3-trione;
5-[(3S)-5-fluoro-7-hydroxy-3-({[4-(trifluoromethyl)cyclohexyl]methyl}amino)-3,4-dihydro-2H-1-benzothiopyran-6-yl]-1λ⁶,2,5-thiadiazolidine-1,1,3-trione;
5-[(3S)-5-fluoro-7-hydroxy-3-{[2-(2,6,6-trimethylcyclohex-1-en-1-yl)ethyl]amino}-3,4-dihydro-2H-1-benzothiopyran-6-yl]-1λ⁶,2,5-thiadiazolidine-1,1,3-trione; and
tert-butyl 4-({[(3S)-5-fluoro-7-hydroxy-6-(1,1,4-trioxo-1λ⁶,2,5-thiadiazolidin-2-yl)-3,4-dihydro-2H-1-benzothiopyran-3-yl]amino}methyl)piperidine-1-carboxylate,
   or a pharmaceutically acceptable salt thereof.

In some embodiments, a compound disclosed herein is formulated as a pharmaceutically acceptable composition comprising a disclosed compound and a pharmaceutically acceptable carrier. In some embodiments, disclosed herein is a method of treating non-small cell lung cancer in a patient in need thereof comprising administering to the patient an effective amount of a compound disclosed herein.

Table of Exemplary Compounds

| No. | Structure |
|---|---|
| 100 | 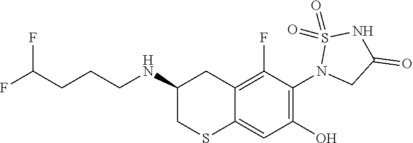 [SMILES: O=C1CN(c2c(O)cc3c(c2F)C[C@H](NCCCC(F)F)CS3)S(=O)(=O)N1] |
| 101 | 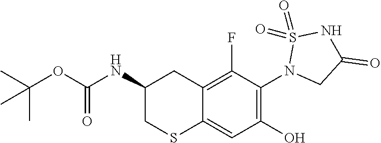 [SMILES: CC(C)(C)OC(=O)N[C@@H]1CSc2cc(O)c(N3CC(=O)NS3(=O)=O)c(F)c2C1] |
| 102 | 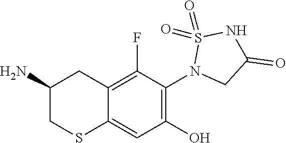 [SMILES: N[C@@H]1CSc2cc(O)c(N3CC(=O)NS3(=O)=O)c(F)c2C1] |
| 103 | 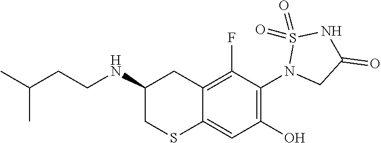 [SMILES: CC(C)CCN[C@@H]1CSc2cc(O)c(N3CC(=O)NS3(=O)=O)c(F)c2C1] |
| 104 | 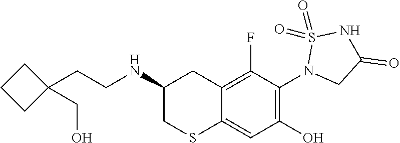 [SMILES: O=C1CN(c2c(O)cc3c(c2F)C[C@H](NCCC2(CO)CCC2)CS3)S(=O)(=O)N1] |
| 105 | 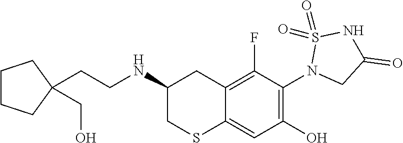 [SMILES: O=C1CN(c2c(O)cc3c(c2F)C[C@H](NCCC2(CO)CCCC2)CS3)S(=O)(=O)N1] |
| 106 | 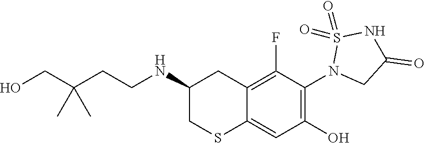 [SMILES: CC(C)(CO)CCN[C@@H]1CSc2cc(O)c(N3CC(=O)NS3(=O)=O)c(F)c2C1] |
| 107 | 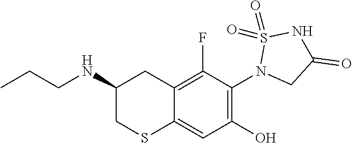 [SMILES: CCCN[C@@H]1CSc2cc(O)c(N3CC(=O)NS3(=O)=O)c(F)c2C1] |
| 108 | 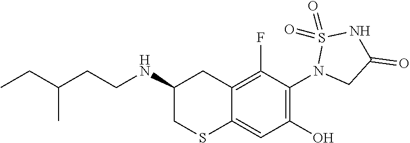 [SMILES: CCC(C)CCN[C@@H]1CSc2cc(O)c(N3CC(=O)NS3(=O)=O)c(F)c2C1] |
| 109 | 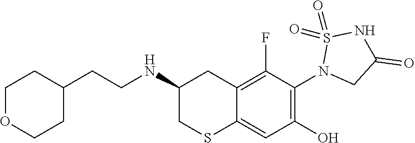 [SMILES: O=C1CN(c2c(O)cc3c(c2F)C[C@H](NCCC2CCOCC2)CS3)S(=O)(=O)N1] |
| 110 | 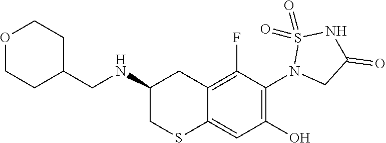 [SMILES: O=C1CN(c2c(O)cc3c(c2F)C[C@H](NCC2CCOCC2)CS3)S(=O)(=O)N1] |
| 111 | 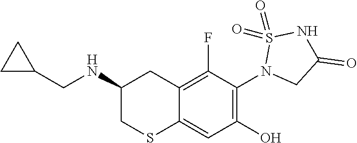 [SMILES: O=C1CN(c2c(O)cc3c(c2F)C[C@H](NCC2CC2)CS3)S(=O)(=O)N1] |
| 112 | 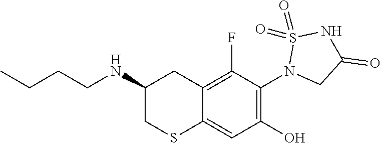 [SMILES: CCCCN[C@@H]1CSc2cc(O)c(N3CC(=O)NS3(=O)=O)c(F)c2C1] |
| 113 | 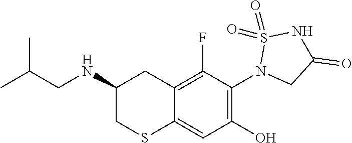 [SMILES: CC(C)CN[C@@H]1CSc2cc(O)c(N3CC(=O)NS3(=O)=O)c(F)c2C1] |
| 114 | 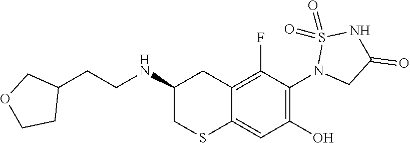 [SMILES: O=C1CN(c2c(O)cc3c(c2F)C[C@H](NCCC2CCOC2)CS3)S(=O)(=O)N1] |
| 115 | 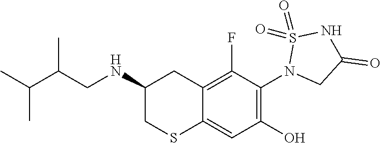 [SMILES: CC(C)C(C)CN[C@@H]1CSc2cc(O)c(N3CC(=O)NS3(=O)=O)c(F)c2C1] |
| 116 | 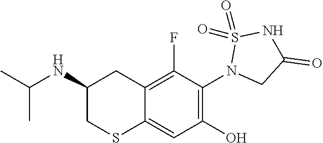 [SMILES: CC(C)N[C@@H]1CSc2cc(O)c(N3CC(=O)NS3(=O)=O)c(F)c2C1] |
| 117 | 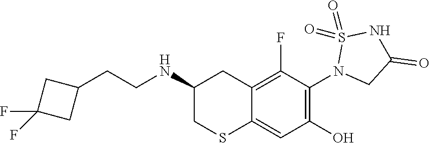 [SMILES: O=C1CN(c2c(O)cc3c(c2F)C[C@H](NCCC2CC(F)(F)C2)CS3)S(=O)(=O)N1] |
| 118 | 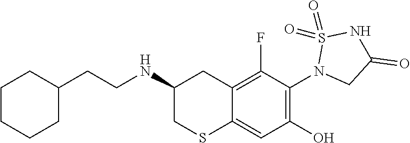 [SMILES: O=C1CN(c2c(O)cc3c(c2F)C[C@H](NCCC2CCCCC2)CS3)S(=O)(=O)N1] |
| 119 | 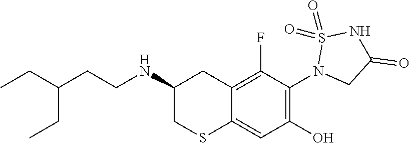 [SMILES: CCC(CC)CCN[C@@H]1CSc2cc(O)c(N3CC(=O)NS3(=O)=O)c(F)c2C1] |
| 120 | 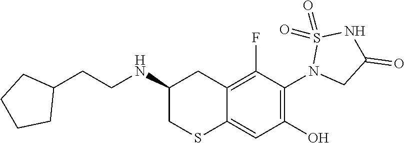 [SMILES: O=C1CN(c2c(O)cc3c(c2F)C[C@H](NCCC2CCCC2)CS3)S(=O)(=O)N1] |
| 121 | 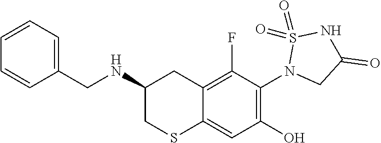 [SMILES: O=C1CN(c2c(O)cc3c(c2F)C[C@H](NCc2ccccc2)CS3)S(=O)(=O)N1] |
| 122 | 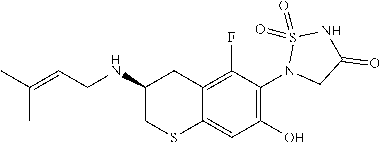 [SMILES: CC(C)=CCN[C@@H]1CSc2cc(O)c(N3CC(=O)NS3(=O)=O)c(F)c2C1] |
| 123 | 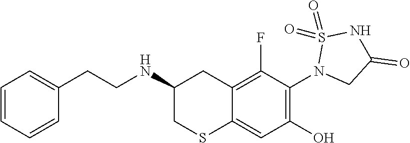 [SMILES: O=C1CN(c2c(O)cc3c(c2F)C[C@H](NCCc2ccccc2)CS3)S(=O)(=O)N1] |
| 124 | 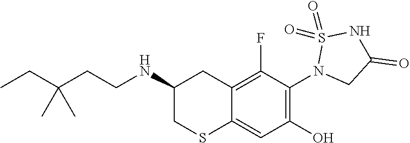 [SMILES: CCC(C)(C)CCN[C@@H]1CSc2cc(O)c(N3CC(=O)NS3(=O)=O)c(F)c2C1] |
| 125 | 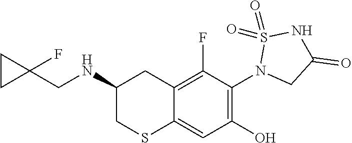 [SMILES: O=C1CN(c2c(O)cc3c(c2F)C[C@H](NCC2(F)CC2)CS3)S(=O)(=O)N1] |
| 126 | 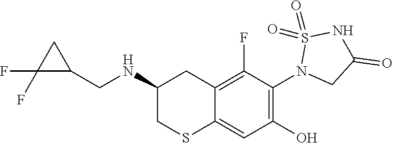 [SMILES: O=C1CN(c2c(O)cc3c(c2F)C[C@H](NCC2CC2(F)F)CS3)S(=O)(=O)N1] |
| 127 | 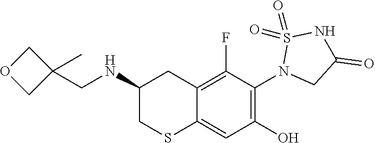 [SMILES: CC1(CN[C@@H]2CSc3cc(O)c(N4CC(=O)NS4(=O)=O)c(F)c3C2)COC1] |
| 128 | 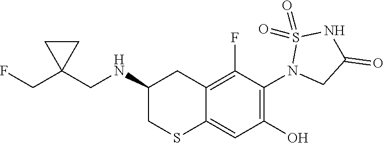 [SMILES: O=C1CN(c2c(O)cc3c(c2F)C[C@H](NCC2(CF)CC2)CS3)S(=O)(=O)N1] |
| 129 | 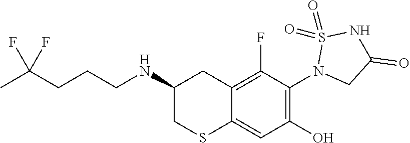 [SMILES: CC(F)(F)CCCN[C@@H]1CSc2cc(O)c(N3CC(=O)NS3(=O)=O)c(F)c2C1] |
| 130 | 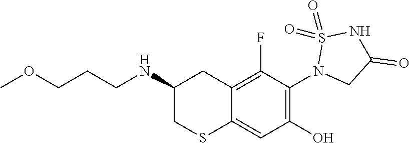 [SMILES: COCCCN[C@@H]1CSc2cc(O)c(N3CC(=O)NS3(=O)=O)c(F)c2C1] |
| 131 | 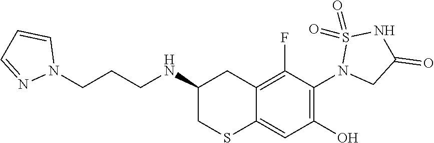 [SMILES: O=C1CN(c2c(O)cc3c(c2F)C[C@H](NCCCn2cccn2)CS3)S(=O)(=O)N1] |
| 132 | 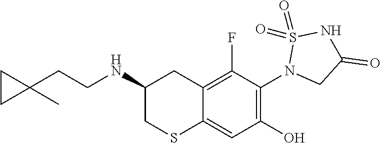 [SMILES: CC1(CCN[C@@H]2CSc3cc(O)c(N4CC(=O)NS4(=O)=O)c(F)c3C2)CC1] |
| 133 | 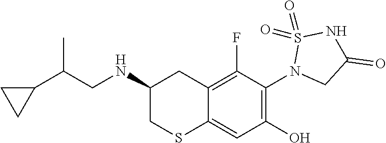 [SMILES: CC(CN[C@@H]1CSc2cc(O)c(N3CC(=O)NS3(=O)=O)c(F)c2C1)C1CC1] |
| 134 | 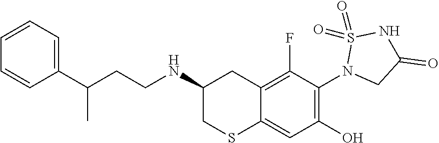 [SMILES: CC(CCN[C@@H]1CSc2cc(O)c(N3CC(=O)NS3(=O)=O)c(F)c2C1)c1ccccc1] |
| 135 | 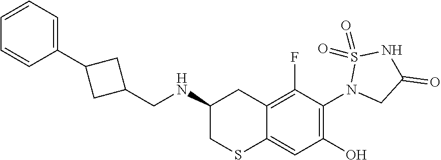 [SMILES: O=C1CN(c2c(O)cc3c(c2F)C[C@H](NCC2CC(c4ccccc4)C2)CS3)S(=O)(=O)N1] |
| 136 | 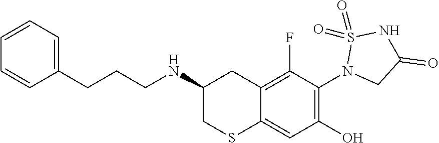 [SMILES: O=C1CN(c2c(O)cc3c(c2F)C[C@H](NCCCc2ccccc2)CS3)S(=O)(=O)N1] |
| 137 | 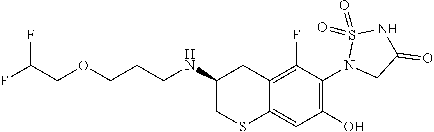 [SMILES: O=C1CN(c2c(O)cc3c(c2F)C[C@H](NCCCOCC(F)F)CS3)S(=O)(=O)N1] |
| 138 | 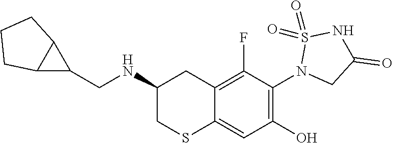 [SMILES: O=C1CN(c2c(O)cc3c(c2F)C[C@H](NCC2C4CCCC42)CS3)S(=O)(=O)N1] |
| 139 | 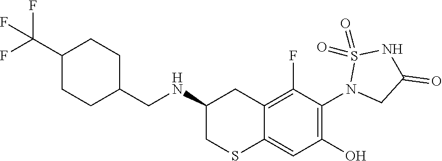 [SMILES: O=C1CN(c2c(O)cc3c(c2F)C[C@H](NCC2CCC(C(F)(F)F)CC2)CS3)S(=O)(=O)N1] |
| 140 | 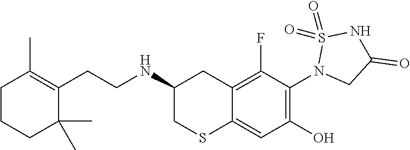 [SMILES: CC1=C(CCN[C@@H]2CSc3cc(O)c(N4CC(=O)NS4(=O)=O)c(F)c3C2)C(C)(C)CCC1] |
| 141 | 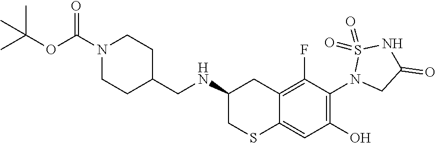 [SMILES: CC(C)(C)OC(=O)N1CCC(CN[C@@H]2CSc3cc(O)c(N4CC(=O)NS4(=O)=O)c(F)c3C2)CC1] |

### Methods of Making Exemplary Compounds

The compounds of the present disclosure may be better understood in connection with the following synthetic schemes and methods which illustrate a means by which the compounds can be prepared. The compounds of the present disclosure can be prepared by a variety of synthetic procedures. Representative synthetic procedures are shown in, but not limited to, Scheme 1.

### Scheme 1: Representative Scheme for Synthesis of Exemplary Compounds of the Disclosure.

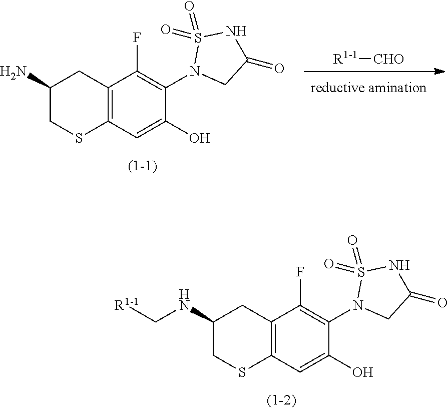

Compounds of formula (1-2) can be reductively aminated with aldehydes, R¹⁻¹—CHO, wherein R¹⁻¹ is optionally substituted C₁₋₆alkyl, optionally substituted C₁₋₆alkenyl, optionally substituted 3-7-membered heteroalkyl, optionally substituted —C₁₋₅alkylene-C₃₋₆cycloalkyl, optionally substituted —C₁₋₅alkylene-C₃₋₆cycloalkenyl, optionally substituted —C₁₋₅alkylene-C₆₋₁₀aryl, optionally substituted —C₁₋₅alkylene-4-6 membered heteroaryl or optionally substituted —C₁₋₅alkylene-4-6 membered heterocyclyl, to give compounds of formula (1-2). Compounds of formula (1-1) can be combined with aldehydes, R¹⁻¹—CHO in the presence of a tertiary amine such as triethylamine in a solvent such as ethanol or a mixture of ethanol and dichloromethane. Subsequent treatment with a reductant such as sodium borohydride gives compounds of formula (1-2). Compounds of formula (1-2) are representative of compounds of Formula (I).

### Pharmaceutical Compositions

The present disclosure provides pharmaceutical compositions comprising a compound disclosed herein, e.g., a compound of Formula (I). In some embodiments, the pharmaceutical composition further comprises a pharmaceutically acceptable excipient. In some embodiments, a compound disclosed herein, e.g., a compound of Formula (I), is provided in an effective amount in the pharmaceutical composition. In some embodiments, the effective amount is a therapeutically effective amount. In certain embodiments, the effective amount is a prophylactically effective amount.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting the activity of a target molecule (e.g. PTPN2 and/or PTPN1), and/or reducing, eliminating, or slowing the progression of disease symptoms. Determination of a therapeutically effective amount of a compound disclosed herein is well within the capabilities of those skilled in the art, especially in light of the detailed disclosure herein.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 20 carbon atoms (“C₁-C₂N alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₆ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present disclosure. The term “alkenylene,” by itself or as part of another substituent, means, unless otherwise stated, a divalent radical derived from an alkene. An alkylene group may be described as, e.g., a C₁-C₆-membered alkylene, wherein the term “membered” refers to the non-hydrogen atoms within the moiety.

“Alkenyl” refers to a radical of a straight-chain or branched hydrocarbon group having from 2 to 20 carbon atoms, one or more carbon-carbon double bonds, and no triple bonds (“C₂-C₂₀ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

The term “alkoxy” refers to an —O-alkyl radical, wherein the alkyl residues is as defined above, and which is attached via an oxygen atom.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

Additionally, terms such as “haloalkyl” are meant to include monohaloalkyl and polyhaloalkyl. For example, the term “halo-C₁-C₆ alkyl” includes, but is not limited to, fluoromethyl, difluoromethyl, trifluoromethyl, 2,2,2-trifluoroethyl, 4-chlorobutyl, 3-bromopropyl, and the like.

“Heteroaryl” refers to a radical of a 5-10 membered monocyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl.

Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl.

“Cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), bicyclo[1.1.1]pentanyl (C₅), bicyclo[2.2.2]octanyl (C₈), bicyclo[2.1.1]hexanyl (C₆), bicyclo[3.1.1]heptanyl (C₇), bicyclo[3.1.0]hexanyl and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

In some embodiments, “cycloalkyl” is a monocyclic, saturated cycloalkyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is substituted C₃-C₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, 3,4-dihydro-2H-1-benzothiopyran, and the like.

“Hydroxy” or “hydroxyl” refers to the radical —OH.

In some embodiments one or more of the nitrogen atoms of a disclosed compound if present are oxidized to the corresponding N-oxide.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

### Other Definitions

“Treating” or “treatment” refers to a method of alleviating or abrogating a disease and/or its attendant symptoms. In certain embodiments, the compounds disclosed herein are useful in the treatment of non-small cell lung cancer. In certain embodiments, a method of treating a human subject with non-small cell lung cancer comprising administering to the human subject in need thereof a therapeutically effective amount of a compound of formula (I) is provided.

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered for treatment in a particular subject or subject population. The term “subject” as used herein, refers to a human. The terms “human,” patient,” and “subject” are used interchangeably herein.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor.

“Patient” or “subject” in need thereof refers to a living organism suffering from or prone to a disease or condition that can be treated by administration of a compound or pharmaceutical composition, as provided herein. In some embodiments, a patient is human.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient.

The term “PTPN2” as used herein refers to protein tyrosine phosphatase non-receptor type 2. The term “PTPN1” refers to protein tyrosine phosphatase non-receptor type 1 (PTPN1), also known as protein tyrosine phosphatase-1B (PTP1B),

In some embodiments, a compound disclosed herein is formulated as a pharmaceutically acceptable composition comprising a disclosed compound and a pharmaceutically acceptable carrier.

### EXEMPLIFICATION

In order that the present disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General schemes relating to methods of making exemplary compounds of the present disclosure are additionally described in the section entitled Methods of Making Exemplary Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization; DMSO for dimethyl sulfoxide; ESI for electrospray ionization; HPLC for high performance liquid chromatography; MS for mass spectrum; NMR for nuclear magnetic resonance; ppm for parts per million; and v/v for volume/volume.

### Example 1: 5-{(3S)-3-[(4,4-difluorobutyl)amino]-5-fluoro-7-hydroxy-3,4-dihydro-2H-1-benzothiopyran-6-yl}-1λ⁶,2,5-thiadiazolidine-1,1,3-trione (Compound 100)

Triethylamine (0.06 mL, 0.45 mmol, 3.0 equivalents) and a solution of 4,4-difluorobutanal in dichloromethane (˜25% weight, 193 mg, 0.45 mmol, 3.0 equivalents) were added in sequence to a suspension of the product of Example 3P (nominally 0.15 mmol, 1 equivalent) in 50% ethanol-dichloromethane (0.80 mL, ˜0.2 M) at 23° C. The reaction mixture was stirred for 2 hours at 23° C. Sodium borohydride (22.5 mg, 0.60 mmol, 4.0 equivalents) was added at 23° C. The reaction mixture was stirred for 20 minutes at 23° C. The mixture was diluted with aqueous hydrochloric acid (3 M, 0.20 mL; CAUTION: gas evolution!), water (1.0 mL) and dimethyl sulfoxide (1.0 mL). The diluted mixture was partially concentrated. The partially concentrated mixture was purified by preparative high-performance liquid chromatography (Phenomenex® C8(2) Luna® 5 μm, AXIA™ 30×75 mm [3 columns coupled], elution with a gradient of 5-100% 0.1% trifluoroacetic acid-acetonitrile-water [v/v]) to furnish the title compound (19 mg, 24% over 3 steps). ¹H NMR (600 MHz, DMSO-d₆) δ ppm 9.65 (s, 1H), 8.77 (s, 1H), 8.59 (s, 1H), 6.48 (s, 1H), 6.15 (tt, J=56.6, 4.2 Hz, 1H), 3.95 (s, 2H), 3.82 (bs, 1H), 3.17-3.06 (m, 4H), 2.75 (dd, J=16.8, 7.9 Hz, 1H), 1.99-1.87 (m, 2H), 1.81-1.71 (m, 2H); MS (APCI⁻) m/z 424 [M−H]⁻.

### Example 2: tert-butyl [(3S)-5-fluoro-7-hydroxy-6-(1,1,4-trioxo-1λ⁶,2,5-thiadiazolidin-2-yl)-3,4-dihydro-2H-1-benzothiopyran-3-yl]carbamate (Compound 101)

### Example 2A: methyl ({(3S)-7-(benzyloxy)-3-[(tert-butoxycarbonyl)amino]-5-fluoro-3,4-dihydro-2H-1-benzothiopyran-6-yl}amino)acetate

The title compound was prepared in 95% yield from the product of Example 3D using the procedure described for Example 3H. ¹H NMR (DMSO-d₆, 400 MHz) δ ppm 7.29-7.48 (m, 5H), 7.01-7.08 (m, 1H), 6.55 (s, 1H), 5.08 (s, 2H), 4.87-4.94 (m, 1H), 4.00-4.06 (m, 1H), 3.74-3.86 (m, 1H), 3.74-3.86 (m, 1H), 3.74-3.86 (m, 1H), 3.59 (s, 3H), 2.93 (br d, J=11.6 Hz, 1H), 2.76-2.87 (m, 2H), 2.42-2.48 (m, 1H), 1.40 (s, 9H).

### Example 2B: methyl [{(3S)-7-(benzyloxy)-3-[(tert-butoxycarbonyl)amino]-5-fluoro-3,4-dihydro-2H-1-benzothiopyran-6-yl}({[(prop-2-en-1-yl)oxy]carbonyl}sulfamoyl)amino]acetate

The title compound was prepared from the product of Example 2A in 92% yield using the procedure described for Example 3I. MS (ESI⁺) m/z 584 [M+H]⁺.

### Example 2C: tert-butyl [(3S)-7-(benzyloxy)-5-fluoro-6-(1,1,4-trioxo-1λ⁶2,5-thiadiazolidin-2-yl)-3,4-dihydro-2H-1-benzothiopyran-3-yl]carbamate

To a solution of the product of Example 2B (3 g, 4.69 mmol) in methanol (90 mL) was added tetrakis(triphenylphosphine)palladium(0) (0.542 g, 0.469 mmol) and potassium carbonate (1.944 g, 14.07 mmol) at 25° C. under nitrogen, and the mixture was stirred at 50° C. for 6 hours under nitrogen. One additional reaction on 200 mg and one on 500 mg scale were run as described above, respectively. The reaction mixtures were combined, acidified with aqueous hydrochloric acid (1 mol/L) to pH=5-6, and then filtered. The filtrate was diluted with water (300 mL) and extracted with ethyl acetate (3×40 mL). The combined organic fractions were washed with brine (50 mL), dried over anhydrous sodium sulfate, filtered, and concentrated under reduced pressure. The residue was purified by preparative HPLC on a Welch Xtimate® C18 column (250×50 mm×10 μm) eluting with acetonitrile (A)—10 mM NH₄HCO₃ in H₂O (gradient 20-55% of A in 30 minutes) at a flow rate of 80 mL/minute to give the title compound (1.7 g, purity 95%, yield 53.5%). ¹H NMR (400 MHz, methanol-d₄) δ ppm 7.49 (d, J=7.34 Hz, 2H), 7.31-7.37 (m, 2H), 7.24-7.30 (m, 1H), 6.66 (d, J=1.34 Hz, 1H), 5.11 (s, 2H), 4.17-4.30 (m, 2H), 4.05 (br s, 1H), 3.05-3.11 (m, 1H), 2.90-3.01 (m, 2H), 2.65 (dd, J=16.69, 8.74 Hz, 1H), 1.45 (s, 9H); MS (ESI⁻) m/z 522 [M−H]⁻.

### Example 2D: tert-butyl [(3S)-5-fluoro-7-hydroxy-6-(1,1,4-trioxo-1λ⁶,2,5-thiadiazolidin-2-yl)-3,4-dihydro-2H-1-benzothiopyran-3-yl]carbamate

A suspension of the product of Example 2C (7 mg, 0.013 mmol), 20% dihydroxypalladium on carbon (18.18 mg, 0.129 mmol) and ammonium formate (16.33 mg, 0.259 mmol) in ethanol (2 mL) was stirred at 60° C. for 2 hours. The reaction mixture was filtered through a plug of diatomaceous earth. The filter cake was washed with methanol (3×1.0 mL). The filtrate and washes were combined and concentrated. The residue was purified by preparative HPLC on a Phenomenex® Luna® 10 μm C18 column (30 mm×250 mm) eluting with a gradient of acetonitrile (A) and water with 0.10% trifluoroacetic acid (B) at a flow rate of 50 mL/minute (0-1 minute 10% A, 1-30 minutes linear gradient 10-95%) to give the title compound (3 mg, 54% yield). ¹H NMR (400 MHz, methanol-d₄) δ ppm 6.37 (d, J=1.7 Hz, 1H), 4.17 (s, 2H), 3.93 (s, 1H), 2.96 (d, J=12.6 Hz, 1H), 2.88-2.78 (m, 2H), 2.51 (dd, J=16.7, 8.9 Hz, 1H), 1.35 (s, 9H); MS (APCI⁻) m/z 432.1 [M−H]⁻.

### Example 3: 5-[(3S)-3-amino-5-fluoro-7-hydroxy-3,4-dihydro-2H-1-benzothiopyran-6-yl]-1λ⁶,2,5-thiadiazolidine-1,1,3-trione (Compound 102)

### Example 3A: 1-(benzyloxy)-5-bromo-3-fluoro-2-nitrobenzene

A solution of potassium tert-butoxide in tetrahydrofuran (1.0 M, 44.0 mL, 44.0 mmol, 1.1 equivalents) was added slowly down the side of the reaction vessel to a suspension of benzyl alcohol (4.60 mL, 44.2 mmol, 1.1 equivalents) and 5-bromo-1,3-difluoro-2-nitrobenzene (10.0 g, 42.0 mmol, 1 equivalent) in tetrahydrofuran (200 mL, 0.21 M) at −67° C. at a rate such that the internal temperature did not exceed −57° C. The reaction mixture was stirred for 5 minutes such that the internal temperature did not exceed −57° C. The product mixture was then diluted sequentially with saturated aqueous ammonium chloride solution (10 mL), water (50 mL), and ethyl acetate (100 mL) at −78° C. The diluted product mixture was warmed over 30 minutes to 23° C. The resulting biphasic mixture was then transferred to a separatory funnel and the layers that formed were separated. The aqueous layer was extracted with ethyl acetate (50 mL). The organic layers were combined and the combined organic layers were washed with saturated aqueous sodium chloride solution (50 mL). The washed organic layer was dried over sodium sulfate. The dried solution was filtered and the filtrate was concentrated. The residue obtained was precipitated from heptanes (125 mL). The mother liquor was concentrated and the residue obtained was triturated with heptanes (3×25 mL). The triturated material was combined with previously precipitated material. The combined material was dried in a vacuum oven at 50° C. to furnish the title compound (11.3 g, 82%). ¹H NMR (400 MHz, DMSO-d₆) δ ppm 7.63 (t, J=1.7 Hz, 1H), 7.57 (dd, J=9.3, 1.7 Hz, 1H), 7.46-7.32 (m, 5H), 5.36 (s, 2H).

### Example 3B: 2-(benzyloxy)-4-bromo-6-fluoroaniline

Saturated aqueous ammonium chloride solution (60 mL, 5.3 volume equivalents) was added to a suspension of zinc powder (11.3 g, 173 mmol, 5.0 equivalents) and the product of Example 3A (11.3 g, 34.7 mmol, 1 equivalent) in 50% methanol-tetrahydrofuran mixture (v/v, 230 mL, 0.15 M) in an ice bath at a rate such that the internal temperature did not exceed 31° C. The reaction vessel was then removed from its cooling bath and warmed over 5 hours to 23° C. Diatomaceous earth (21 g) was added to the product mixture, and the resulting slurry was stirred for 10 minutes at 23° C. The mixture was then filtered through a plug of diatomaceous earth (2.0 cm×8.0 cm). The filter cake was washed with methanol (3×30 mL). The filtrate and washes were combined and concentrated. The residue obtained was dissolved in ethyl acetate (200 mL), and the mixture was washed successively with water (50 mL) and brine (50 mL). The washed organic layer was dried over sodium sulfate. The dried solution was filtered, and the filtrate was concentrated. The residue obtained was used without further purification in the following step. MS (ESI⁺) m/z 296 [M+H]⁺.

### Example 3C: N-[2-(benzyloxy)-4-bromo-6-fluorophenyl]-2,2,2-trifluoroacetamide

Trifluoroacetic anhydride (5.90 mL, 41.8 mmol, 1.2 equivalents) was added over 10 minutes via syringe pump to a solution of the product of Example 3B (nominally 34.7 mmol, 1 equivalent) in dichloromethane (174 mL, 0.2 M) at 0° C. such that the internal temperature did not exceed 7° C. The reaction mixture was warmed over 18 hours to 23° C. The product mixture was partitioned between water (50 mL) and ethyl acetate (500 mL). The organic layer was washed sequentially with hydrochloric acid solution (1 M, 3×100 mL) and saturated aqueous sodium chloride solution (100 mL). The washed organic layer was dried over sodium sulfate. The dried solution was filtered. Diatomaceous earth (˜50 g) was added to the filtrate, and the mixture was concentrated. The residue obtained was purified by flash column chromatography (330 g Teledyne ISCO RediSep Rf Gold® silica column, elution with a gradient from 0-100% ethyl acetate-heptanes) to furnish the title compound (9.18 g, 67%, 2 steps). MS (ESI⁻) m/z 390 [M−H]⁻.

### Example 3D: tert-butyl [(2S)-1-[4-(benzyloxy)-6-bromo-2-fluoro-3-(2,2,2-trifluoroacetamido)phenyl]-3-{[tert-butyl(dimethyl)silyl]oxy}propan-2-yl]carbamate

A solution of n-butyllithium in hexanes (1.91 M, 5.50 mL, 10.50 mmol, 2.1 equivalents) was added to a solution of diisopropylamine (1.57 mL, 11.00 mmol, 2.2 equivalents) in tetrahydrofuran (20.0 mL) at −78° C. The mixture was stirred for 15 minutes at −78° C. A solution of the product of Example 3C (2.06 g, 5.25 mmol, 1.05 equivalents) in tetrahydrofuran (6.5 mL) was added dropwise via syringe pump over 20 minutes at −78° C. The mixture was stirred for 30 minutes at −78° C. A solution of tert-butyl (4R)-4-({[tert-butyl(dimethyl)silyl]oxy}methyl)-2,2-dioxo-1,2λ⁶,3-oxathiazolidine-3-carboxylate (1.84 g, 5.00 mmol, 1 equivalent; Tetrahedron Lett. 2011, 52, 5229-5233) in tetrahydrofuran (6.5 mL; 0.15 M overall) was added dropwise over 20 minutes at −78° C. The reaction mixture was stirred for 30 minutes at −78° C. Aqueous hydrochloric acid solution (3 M, 8.33 mL, 25.00 mmol, 5.0 equivalents) was added at −78° C. The resulting mixture was warmed over 20 minutes to 23° C. The warmed mixture was diluted with ethyl acetate (100 mL). The resulting biphasic mixture was transferred to a separatory funnel and the layers that formed were separated. The aqueous layer was extracted with ethyl acetate (50 mL). The organic layers were combined and the combined organic layers were washed with saturated aqueous sodium chloride solution (20 mL). The washed organic layer was dried over sodium sulfate. The dried solution was filtered and the filtrate was concentrated. The residue obtained was dissolved in ether (20 mL). Diatomaceous earth (˜10 g) was added to the solution and the mixture was concentrated. The residue obtained was purified by flash column chromatography (80 g Teledyne ISCO RediSep Rf Gold® silica column, elution with a gradient from 0-80% ethyl acetate-heptanes) to furnish the title compound (1.977 g, 58%). MS (APCI⁺) m/z 579 [M+H−C(O)OC(CH₃)₃]⁺.

### Example 3E: methyl [{6-(benzyloxy)-4-bromo-3-[(2S)-2-[(tert-butoxycarbonyl)amino]-3-{[tert-butyl(dimethyl)silyl]oxy}propyl]-2-fluorophenyl}(trifluoroacetyl)amino]acetate

Methyl bromoacetate (0.22 mL, 2.43 mmol, 1.1 equivalents) was added to a suspension of the product of Example 3D (1.5 g, 2.21 mmol, 1 equivalent), potassium carbonate (915 mg, 6.62 mmol, 3.0 equivalents), and potassium iodide (183 mg, 1.10 mmol, 0.5 equivalent) in acetone (11 mL, 0.2 M) at 23° C. The reaction mixture was stirred for 24 hours at 23° C. The mixture was concentrated. The residue obtained was partitioned between ethyl acetate (60 mL) and water (15 mL). The aqueous layer was extracted with ethyl acetate (30 mL). The organic layers were combined and washed with saturated aqueous sodium chloride solution (15 mL). The washed organic layer was dried over sodium sulfate. The dried solution was filtered and the filtrate was concentrated. The titled compound obtained was used without further purification in the following step. MS (APCI⁺) m/z 651 [M+H−C(O)OC(CH₃)₃]⁺.

### Example 3F: methyl {[6-(benzyloxy)-4-bromo-3-{(2S)-2-[(tert-butoxycarbonyl)amino]-3-hydroxypropyl}-2-fluorophenyl](trifluoroacetyl)amino}acetate

A solution of tetrabutylammonium fluoride in tetrahydrofuran (1.0 M, 13.3 mL, 13.3 mmol, 1.1 equivalents) was added to a solution of the product of Example 3E (9.07 g, 12.1 mmol, 1 equivalent) in tetrahydrofuran (60.0 mL, 0.2 M) at 23° C. The reaction mixture was stirred for 3 days at 23° C. The reaction mixture was partitioned between saturated aqueous ammonium chloride solution (15 mL), water (15 mL), and ethyl acetate (200 mL). The organic layer was separated and then washed with saturated aqueous sodium chloride solution (20 mL). The washed organic layer was dried over sodium sulfate and then filtered. Diatomaceous earth (˜40 g) was added to the filtrate, and the mixture was concentrated. The residue obtained was purified by flash column chromatography (120 g Teledyne ISCO RediSep Rf Gold® silica column, elution with a gradient from 0-100% ethyl acetate-heptanes) to furnish the title compound (7.15 g, 93%). MS (APCI⁺) m/z 637 [M+H]⁺.

### Example 3G: methyl {[6-(benzyloxy)-4-bromo-3-{(2S)-2-[(tert-butoxycarbonyl)amino]-3-[(methanesulfonyl)oxy]propyl}-2-fluorophenyl](trifluoroacetyl)amino}acetate

Methanesulfonyl chloride (1.05 mL, 13.46 mmol, 1.2 equivalents) was added to a solution of the product of Example 3F (7.15 g, 11.22 mmol, 1 equivalent) and N,N-diisopropylethylamine (3.92 mL, 22.43 mmol, 2.0 equivalents) in dichloromethane (56 mL, 0.2 M) at 0° C. The reaction mixture was warmed over 12 hours to 23° C. Diatomaceous earth (˜30 g) was added to the reaction mixture, and the mixture was concentrated. The residue obtained was purified by flash column chromatography (80 g Teledyne ISCO RediSep Rf Gold® silica column, elution with a gradient from 0-100% ethyl acetate-heptanes). The fractions containing product were combined and concentrated. The residue obtained (7.13 g, <89%) was used without further purification in the following step. MS (APCI⁺) m/z 615 [M+H−C(O)OC(CH₃)₃]⁺.

### Example 3H: methyl {[3-{(2S)-3-(acetylsulfanyl)-2-[(tert-butoxycarbonyl)amino]propyl}-6-(benzyloxy)-4-bromo-2-fluorophenyl](trifluoroacetyl)amino}acetate

Potassium thioacetate (1.59 g, 13.95 mmol, 1.4 equivalents) was added to a solution of the product of Example 3G (7.13 g, 9.96 mmol, 1 equivalent) in N,N-dimethylformamide (50 mL, 0.2 M) at 23° C. The reaction mixture was stirred for 16 hours at 23° C. The reaction mixture was partially concentrated. The residue obtained was partitioned between ethyl acetate (150 mL), water (15 mL), and saturated aqueous ammonium chloride solution (10 mL). The aqueous layer was extracted with ethyl acetate (75 mL). The organic layers were combined and the combined organic layers were washed with saturated aqueous sodium chloride solution (3×10 mL). The washed organic layer was dried over sodium sulfate. The dried solution was filtered. Diatomaceous earth (˜40 g) was added to the filtrate, and the mixture was concentrated. The residue obtained was purified by flash column chromatography (120 g Teledyne ISCO RediSep Rf Gold® silica column, elution with a gradient from 0-100% ethyl acetate-heptanes). The fractions containing product were combined and concentrated. The residue obtained (5.30 g, <76%) was used without further purification in the following step. MS (APCI⁺) m/z 595 [M+H−C(O)OC(CH₃)₃]⁺.

### Example 3I: methyl [{(3S)-7-(benzyloxy)-3-[(tert-butoxycarbonyl)amino]-5-fluoro-3,4-dihydro-2H-1-benzothiopyran-6-yl}(trifluoroacetyl)amino]acetate

A suspension of tris(dibenzylideneacetone)dipalladium(0) (70.0 mg, 0.08 mmol, 5.0 mol %), (9,9-dimethyl-9H-xanthene-4,5-diyl)bis(diphenylphosphine) (Xantphos, 89.0 mg, 0.15 mmol, 10.0 mol %), N,N-diisopropylethylamine (0.05 mL, 0.29 mmol, 3.0 equivalents), and the product of Example 3H (63.0 mg, 0.10 mmol, 1 equivalent) in 1,4-dioxane (0.5 mL, ˜0.2 M) was deoxygenated by iterative subjections to vacuum (˜5 seconds) and subsequent backfilling with nitrogen (×3). The reaction vessel was placed in a heating block that had been preheated to 100° C. The reaction mixture was stirred for 45 minutes at 100° C. The product mixture was cooled to 23° C. The reaction mixture was diluted with ethyl acetate (5 mL). Diatomaceous earth (˜1 g) was added to the solution, and the mixture was concentrated. The residue obtained was purified by flash column chromatography (40 g Teledyne ISCO RediSep Rf Gold® silica column, elution with a gradient from 0-100% ethyl acetate-heptanes) to furnish the title compound (1.49 g, 51%). MS (APCI⁺) m/z 573 [M+H]⁺.

### Example 3J: methyl {[(3S)-3-amino-5-fluoro-7-hydroxy-3,4-dihydro-2H-1-benzothiopyran-6-yl](trifluoroacetyl)amino}acetate

A solution of boron trichloride in dichloromethane (41.2 mL, 41.2 mmol, 5.0 equivalents) was added slowly to a solution of the product of Example 3I (4.72 g, 8.24 mmol, 1 equivalent) and pentamethylbenzene (1.34 g, 9.07 mmol, 1.1 equivalents) in dichloromethane (41.2 mL, 0.2 M) at −78° C. The reaction vessel was immediately removed from the cooling bath and warmed over 1 hour to 23° C. The product mixture was then cooled to −78° C. The cooled product mixture was diluted with methanol (35 mL). The diluted mixture was warmed to 23° C. and concentrated. The residue obtained was used without further purification in the following step. MS (APCI⁺) m/z 383 [M+H]⁺.

### Example 3K: methyl [{(3S)-3-[(tert-butoxycarbonyl)amino]-5-fluoro-7-hydroxy-3,4-dihydro-2H-1-benzothiopyran-6-yl}(trifluoroacetyl)amino]acetate

Di-tert-butyl dicarbonate (2.39 g, 10.7 mmol, 1.3 equivalents) was added to a biphasic mixture of the product of Example 3J (nominally 8.24 mmol, 1 equivalent) and sodium bicarbonate (3.46 g, 41.2 mmol, 5.0 equivalents) in 20% water-tetrahydrofuran mixture (v/v, 40.0 mL, ˜0.2 M) at 23° C. The reaction mixture was stirred for 1.5 hours at 23° C. The product mixture was partitioned between ethyl acetate (200 mL) and water (50 mL). The aqueous layer was extracted with ethyl acetate (50 mL). The organic layers were combined and the combined organic layers were washed with saturated aqueous sodium chloride solution (25 mL). The washed organic layer was dried over sodium sulfate. The dried solution was filtered and concentrated. The residue obtained was dissolved in 75% acetone-pentane (50 mL). Diatomaceous earth (˜20 g) was added to the solution and the mixture was concentrated. The residue obtained was purified by flash column chromatography (330 g Teledyne ISCO RediSep Rf Gold® silica column, elution with a gradient from 0-100% ethyl acetate-heptanes) to furnish the title compound (3.81 g, 96%, two steps). ¹H NMR (500 MHz, CDCl₃) δ ppm 8.63 (s, 1H), 6.62 (s, 1H), 5.00 (dd, J=17.4, 3.8 Hz, 1H), 4.97-4.83 (m, 1H), 4.45-4.27 (m, 1H), 3.88 (s, 3H), 3.76-3.61 (m, 1H), 3.19 (ddd, J=29.4, 12.6, 2.1 Hz, 1H), 2.95 (dd, J=12.6, 6.7 Hz, 1H), 2.83-2.75 (m, 2H), 1.44 (s, 9H); MS (APCI⁺) m/z 383 [M+H−C(O)OC(CH₃)₃]⁺.

### Example 3L: methyl [{(3S)-3-[(tert-butoxycarbonyl)amino]-5-fluoro-7-[(2-methoxyethoxy)methoxy]-3,4-dihydro-2H-1-benzothiopyran-6-yl}(trifluoroacetyl)amino]acetate

2-Methoxyethoxymethyl chloride (1.07 mL, 9.48 mmol, 1.2 equivalents) was added to a solution of the product of Example 3K (3.81 g, 7.90 mmol, 1 equivalent) and N,N-diisopropylethylamine (4.14 mL, 23.7 mmol, 3.0 equivalents) in dichloromethane (40.0 mL, 0.2 M) at 0° C. The reaction vessel was immediately removed from its cooling bath and warmed to 23° C. over 1 hour. Additional 2-methoxyethoxymethyl chloride (0.20 mL, 1.77 mmol, 0.22 equivalent) was added at 23° C. The reaction mixture was stirred for 30 minutes at 23° C. The product mixture was partitioned between ethyl acetate (250 mL), heptanes (20 mL), water (10 mL), and saturated aqueous ammonium chloride solution (30 mL). The organic layer was separated and then washed with saturated aqueous sodium chloride solution (30 mL). The washed organic layer was dried over sodium sulfate. The dried solution was filtered and the filtrate was concentrated. The title compound obtained was used without further purification in the following step. MS (APCI⁺) m/z 471 [M+H−C(O)OC(CH₃)₃]⁺.

### Example 3M: methyl ({(3S)-3-[(tert-butoxycarbonyl)amino]-5-fluoro-7-[(2-methoxyethoxy)methoxy]-3,4-dihydro-2H-1-benzothiopyran-6-yl}amino)acetate

A solution of sodium methoxide in methanol (0.5 M, 8.00 mL, 4.00 mmol, 3.0 equivalents) was added to the product of Example 3L (759 mg, 1.33 mmol, 1 equivalent) neat under nitrogen. The reaction vessel was placed in a heating block that had been preheated to 60° C. The reaction mixture was stirred for 1.5 hours at 60° C. The product mixture was then cooled to 23° C. The cooled product mixture was concentrated. The residue obtained was partitioned between aqueous hydrochloric acid solution (1 M, 35 mL) and ethyl acetate (250 mL). The aqueous layer was extracted with ethyl acetate (50 mL). The organic layers were combined and washed with saturated aqueous sodium chloride solution (20 mL). The washed organic layer was dried over sodium sulfate. The dried solution was filtered. Diatomaceous earth (˜20 g) was added to the solution and the mixture was concentrated. The residue obtained was purified by flash column chromatography (220 g Teledyne ISCO RediSep Rf Gold® silica column, elution with a gradient from 0-100% ethyl acetate-heptanes) to furnish the title compound (3.33 g, 89%, two steps). ¹H NMR (600 MHz, CDCl₃) δ ppm 6.69 (s, 1H), 5.25 (s, 2H), 5.04 (bs, 1H), 4.40-431 (m, 2H), 4.04 (dd, J=6.3, 1.2 Hz, 2H), 3.85-3.82 (m, 2H), 3.74 (s, 2H), 3.59-3.55 (m, 2H), 3.40 (s, 3H), 3.14 (d, J=11.8 Hz, 1H), 2.90-2.75 (m, 3H), 1.44 (s, 9H); MS (APCI⁺) m/z 475 [M+H]⁺.

### Example 3N: methyl [{(3S)-3-[(tert-butoxycarbonyl)amino]-5-fluoro-7-[(2-methoxyethoxy)methoxy]-3,4-dihydro-2H-1-benzothiopyran-6-yl}({[(prop-2-en-1-yl)oxy]carbonyl}sulfamoyl)amino]acetate

Allyl alcohol (1.05 mL, 15.4 mmol, 2.2 equivalents) was added to a solution of chlorosulfonyl isocyanate (1.34 mL, 15.4 mmol, 2.2 equivalents) in dichloromethane (20.0 mL) at 0° C. The reaction mixture was stirred for 30 minutes at 0° C. A solution of the product of Example 3M (3.33 g, 7.02 mmol, 1 equivalent) and triethylamine (3.91 mL, 28.1 mmol, 4.0 equivalents) in dichloromethane (15.0 mL, ˜0.2 M overall) was added dropwise at 0° C. The reaction mixture was stirred for 10 minutes at 0° C. The product mixture was partially concentrated. The residue obtained was partitioned between ethyl acetate (75 mL) and water (10 mL). The organic layer was washed with saturated aqueous sodium chloride solution (10 mL). The washed organic layer was dried over sodium sulfate. The dried solution was filtered. Diatomaceous earth (˜15 g) was added to the solution and the mixture was concentrated. The residue obtained was purified by flash column chromatography (120 g Teledyne ISCO RediSep Rf Gold® silica column, elution with a gradient from 0-100% ethyl acetate-heptanes) to furnish the title compound (3.74 g, 84%). MS (APCI⁺) m/z 655 [M+NH₄]⁺.

### Example 3O: tert-butyl [(3S)-5-fluoro-7-[(2-methoxyethoxy)methoxy]-6-(1,1,4-trioxo-1λ⁶2,5-thiadiazolidin-2-yl)-3,4-dihydro-2H-1-benzothiopyran-3-yl]carbamate

A suspension of the product of Example 3N (nominally 0.30 mmol, 1 equivalent), potassium carbonate (122 mg, 0.89 mmol, 3.0 equivalents), and tetrakis(triphenylphosphine)palladium(0) (17.0 mg, 0.02 mmol, 5.0 mol %) in methanol (1.50 mL, 0.2 M) was deoxygenated by iterative subjections to vacuum (˜5 seconds) and subsequent backfilling with nitrogen (×3). The reaction vessel was placed in a heating block that had been preheated to 60° C. The reaction mixture was stirred for 45 minutes at 60° C. The product mixture was then cooled to 23° C. The cooled mixture was filtered through a plug of diatomaceous earth (0.5 cm×1.0 cm). The filter cake was rinsed with ethyl acetate (3×2.0 mL). The filtrates were combined and carefully diluted with aqueous hydrochloric acid solution (1 M, 25 mL). The resulting biphasic mixture was then transferred to a separatory funnel and the layers that formed were separated. The aqueous layer was extracted with ethyl acetate (2×50 mL). The organic layers were combined and the combined organic layers were washed with saturated aqueous sodium chloride solution (15 mL). The washed organic layer was dried over sodium sulfate. The dried solution was filtered and the filtrate was concentrated. The title compound obtained was used without further purification in the following step. ¹H NMR (500 MHz, DMSO-d₆) δ ppm 6.70 (s, 1H), 5.18 (s, 2H), 4.13-4.50 (m, 1H), 3.91-3.77 (m, 3H), 3.72 (dd, J=5.5, 4.0 Hz, 2H), 3.44 (dd, J=5.5, 4.0 Hz, 2H), 3.16 (d, J=4.9 Hz, 2H), 3.01 (d, J=10.6 Hz, 1H), 1.40 (s, 9H); MS (APCI⁺) m/z 539 [M+NH₄]⁺.

### Example 3P: 5-[(3S)-3-amino-5-fluoro-7-hydroxy-3,4-dihydro-2H-1-benzothiopyran-6-yl]-1λ⁶,2,5-thiadiazolidine-1,1,3-trione hydrochloride

A solution of hydrogen chloride in dioxane (4.0 M, 15.3 mL, 61.4 mmol, 20.0 equivalents) was added to a solution of the product of Example 30 (nominally 3.07 mmol, 1 equivalent) and pentamethylbenzene (1.36 g, 9.20 mmol, 3.0 equivalents) in tetrahydrofuran (10.2 mL, 0.3 M) at 23° C. The reaction mixture was stirred for 2.5 hours at 23° C. The product mixture was diluted with ether (25 mL), and a suspension resulted. The mother liquor was decanted. The residue obtained was triturated successively with acetonitrile (2×10 mL), ethyl acetate (5×15 mL), and ether (15 mL). The title compound obtained was used without further purification. ¹H NMR (400 MHz, DMSO-d₆) δ ppm 8.46 (bs, 3H), 6.57 (s, 1H), 4.34 (s, 2H), 3.74 (bs, 1H), 3.26-3.19 (m, 1H), 3.17-3.03 (m, 2H), 2.72 (dd, J=16.6, 8.6 Hz, 1H); MS (APCI⁺) m/z 334 [M+H]⁺.

### Example 4: 5-{(3S)-5-fluoro-7-hydroxy-3-[(3-methylbutyl)amino]-3,4-dihydro-2H-1-benzothiopyran-6-yl}-1λ⁶,2,5-thiadiazolidine-1,1,3-trione (Compound 103)

Isovaleraldehyde (0.02 mL, 0.19 mmol, 3.8 equivalents) was added to a solution of the product of Example 3P (nominally 0.05 mmol, 1 equivalent) and triethylamine (0.04 mL, 0.29 mmol, 5.7 equivalents) in 50% ethanol-dichloromethane (0.25 mL, 0.2 M) at 23° C. The reaction mixture was stirred for 1.5 hours at 23° C. Sodium borohydride (7.2 mg, 0.19 mmol, 3.8 equivalents) was added in one portion at 23° C. The sides of the flask were rinsed with ethanol (0.25 mL). The reaction mixture was stirred for 1 hour at 23° C. The product mixture was concentrated. The residue obtained was dissolved in methanol (1.1 mL) and the solution was purified by high-performance liquid chromatography (Waters XBridge™ RP18 column, 5 μm, 30×100 mm, flow rate 40 mL/minute, 5-100% gradient of acetonitrile in buffer 0.1% trifluoroacetic acid) to furnish the title compound (8.0 mg, 31%, 3 steps). ¹H NMR (600 MHz, DMSO-d₆) δ ppm 9.59 (s, 1H), 8.69 (s, 1H), 8.51 (s, 1H), 6.47 (s, 1H), 3.92 (s, 2H), 3.80 (bs, 1H), 3.19-3.00 (m, 4H), 2.74 (dd, J=16.7, 8.2 Hz, 1H), 1.64 (dq, J=13.3, 6.7 Hz, 1H), 1.54-1.47 (m, 2H), 0.91 (d, J=6.6 Hz, 6H); MS (APCI⁺) m/z 404 [M+H]⁺.

### Example 5: 5-[(3S)-5-fluoro-7-hydroxy-3-({2-[1-(hydroxymethyl)cyclobutyl]ethyl}amino)-3,4-dihydro-2H-1-benzothiopyran-6-yl]-1λ⁶,2,5-thiadiazolidine-1,1,3-trione (Compound 104)

### Example 5A: tert-butyl(dimethyl){[1-(prop-2-en-1-yl)cyclobutyl]methoxy}silane

To a solution of (1-allylcyclobutyl)methanol (prepared according to Bioorganic and Medicinal Chemistry, 2002, 10 (4), 1093-1106) (2.5 g, 15.85 mmol, 80% purity) in anhydrous tetrahydrofuran (70 mL) was added imidazole (2.158 g, 31.7 mmol) and then tert-butyldimethylchlorosilane (3.58 g, 23.77 mmol) in portions at 0° C. The reaction mixture was stirred at 20° C. for 3 hours. One additional reaction on 500 mg scale was set up and run as described above. These two reaction mixtures were combined and diluted with water (200 mL). The organic phase was separated, and the aqueous phase was extracted with ethyl acetate (60 mL). The combined organic phases were washed with brine (100 mL), dried over anhydrous sodium sulfate, and concentrated under reduced pressure. The residue was then dissolved with petroleum ether and filtered through silica gel, and the filter cake was washed with petroleum ether (1500 mL). The filtrate was concentrated under reduced pressure to give the title compound (4 g, 90% purity, 86% yield). ¹H NMR (400 MHz, CDCl₃) δ ppm 5.79 (ddt, J=17.07, 10.07, 7.32 Hz, 1H), 4.96-5.10 (m, 2H), 3.44 (s, 2H), 2.21 (d, J=7.25 Hz, 2H), 1.63-1.92 (m, 6H), 0.92 (s, 9H), −0.05 (s, 6H).

### Example 5B: [1-({[tert-butyl(dimethyl)silyl]oxy}methyl)cyclobutyl]acetaldehyde

To a solution of the product of Example 5A (3 g, 11.23 mmol, purity 90%) in dioxane (120 mL) and water (12 mL) was added a 0.2 M solution osmium tetroxide in t-butanol (220 mg, 0.865 mmol) dropwise at 20° C. After 15 minutes, the reaction mixture was cooled to 0° C. before sodium periodate (9.61 g, 44.9 mmol) was added in portions. After addition, the mixture was warmed up to 20° C. and stirred at that temperature for 3 hours. The mixture was diluted with ethyl acetate (200 mL) and filtered. The filtrate was added to saturated sodium thiosulfate aqueous solution (300 mL) and the resulting mixture was stirred at 20° C. for 1 hour. The mixture was transferred to a separatory funnel and the organic phase was separated. The organic fraction was washed with brine (500 mL), dried over anhydrous sodium sulfate, and concentrated under reduced pressure to give the title compound (3 g, purity 70%, yield 77%) which was used for the next step without further purification. ¹H NMR (400 MHz, CDCl₃) δ ppm 9.63 (t, J=2.75 Hz, 1H), 3.56 (s, 2H), 2.46 (d, J=2.63 Hz, 2H), 1.82-1.90 (m, 6H), 0.89 (s, 9H), 0.02 (s, 6H).

### Example 5C. 5-[(3S)-5-fluoro-7-hydroxy-3-({2-[1-(hydroxymethyl)cyclobutyl]ethyl}amino)-3,4-dihydro-2H-1-benzothiopyran-6-yl]-1λ⁶,2,5-thiadiazolidine-1,1,3-trione

In a 4 mL vial were combined the product of Example 3P (60 mg, 0.162 mmol), and triethylamine (67.8 μL, 0.487 mmol, 3 equivalents) in 3:2 v/v ethanol/dichloromethane (1.6 mL) to give a suspension. Example 5B (78.6 mg, 0.324 mmol, 2.0 equivalents) was added and the reaction mixture was stirred at room temperature for 2 hours. Sodium borohydride (24.5 mg, 0.649 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.6 mL, 3.24 mmol, 20 equivalents) was slowly added, and the reaction mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (27.2 mg, 37.7% yield). ¹H NMR (400 MHz, DMSO-d₆:D₂O=9:1 (v/v)) δ ppm 6.47 (d, J=1.5 Hz, 1H), 3.95 (s, 2H), 3.60 (s, 1H), 3.34 (s, 2H), 3.29-3.22 (m, 1H), 3.11-2.99 (m, 2H), 2.98-2.81 (m, 2H), 2.66 (dd, J=16.8, 8.1 Hz, 1H), 1.89-1.57 (m, 8H); MS (APCI⁺) m/z 446.3 [M+H]⁺.

### Example 6: 5-[(3S)-5-fluoro-7-hydroxy-3-({2-[1-(hydroxymethyl)cyclopentyl]ethyl}amino)-3,4-dihydro-2H-1-benzothiopyran-6-yl]-1λ⁶,2,5-thiadiazolidine-1,1,3-trione (Compound 105)

### Example 6A: methyl 1-(prop-2-en-1-yl)cyclopentane-1-carboxylate

To a solution of 2 M lithium diisopropylamide (122 mL, 243 mmol, tetrahydrofuran) in anhydrous tetrahydrofuran (720 mL) was added methyl cyclopentanecarboxylate (24 g, 187 mmol) dropwise at −65° C. under nitrogen. The reaction mixture was stirred at −65° C. under nitrogen for 40 minutes before 3-bromoprop-1-ene (29.4 g, 243 mmol) was added dropwise. The mixture was then allowed to warm to 20° C. and stirred at 20° C. for 16 hours before it was quenched by adding saturated aqueous NH₄Cl solution (500 mL) dropwise at 0° C. The resulting mixture was extracted with ethyl acetate (3×150 mL). The combined organic phases were washed with brine (500 mL), dried over anhydrous sodium sulfate, filtered and concentrated. The residue was chromatographed on silica gel eluting with petroleum ether to give the title compound (31.2 g, 167 mmol, yield 89%). ¹H NMR (400 MHz, CDCl₃) δ ppm 5.76-5.64 (m, 1H), 5.06-4.98 (m, 2H), 3.67 (s, 3H), 2.34 (d, J=7.3 Hz, 2H), 2.11-2.02 (m, 2H), 1.69-1.58 (m, 4H), 1.58-1.46 (m, 2H).

### Example 6B: [1-(prop-2-en-1-yl)cyclopentyl]methanol

To a solution of the product of Example 6A (15.5 g, 77 mmol, purity 90%) in tetrahydrofuran (300 mL) was added LiAlH₄ (4.65 g, 122 mmol) in portions at 0° C. under nitrogen. Then the mixture was stirred at 0° C. under nitrogen for 2 hours. The reaction mixture was quenched by adding water (4.65 mL) dropwise at 0° C., followed by 15% aqueous NaOH solution (4.65 mL) and water (13.95 mL). One additional same reaction on 15.5 g scale was run as described above. These two reaction mixtures were combined and filtered through a pad of diatomaceous earth. The filter cake was washed with ethyl acetate (1000 mL) and tetrahydrofuran (1000 mL). The filtrate and washes were concentrated under reduced pressure to give the title compound (22.5 g, 144 mmol, yield 94%, purity 90%). ¹H NMR (400 MHz, CDCl₃) δ ppm 5.86 (m, 1H), 5.13-5.03 (m, 2H), 3.40 (s, 2H), 2.17 (d, J=7.4 Hz, 2H), 1.67-1.51 (m, 4H), 1.56-1.50-1.38 (m, 4H).

### Example 6C: tert-butyl(dimethyl){[1-(prop-2-en-1-yl)cyclopentyl]methoxy}silane

To a solution of the product of Example 6B (10.4 g, 74.2 mmol, purity 90%) and imidazole (12.12 g, 178 mmol) in anhydrous dichloromethane (208 mL) was added tert-butyldimethylchlorosilane (12.30 g, 82 mmol) at 0° C. Then the mixture was stirred at 20° C. for 12 hours. Two additional reactions of the same type were run on 2.5 g and 10.4 g scales as described above. These three reaction mixtures were combined, diluted with water (300 mL), and extracted with dichloromethane (3×200 mL). The combined organic fractions were dried over anhydrous sodium sulfate and concentrated under reduced pressure. The residue was purified by column chromatography on silica gel eluted with petroleum ether to give the title compound (39.35 g, 155 mmol, yield 93%, purity 90%). ¹H NMR (400 MHz, CDCl₃) δ ppm 5.87-5.76 (m, 1H), 5.06-4.99 (m, 2H), 3.31 (s, 2H), 2.13 (d, J=7.3 Hz, 2H), 1.61-1.54 (m, 4H), 1.47-1.32 (m, 4H), 0.88 (s, 9H), 0.04 (s, 6H).

### Example 6D: [1-({[tert-butyl(dimethyl)silyl]oxy}methyl)cyclopentyl]acetaldehyde

To a solution of the product of Example 6C (14.5 g, 51.3 mmol, purity 90%) in water (60 mL) and tetrahydrofuran (300 mL) was added a solution of osmium tetroxide (107 mg, 0.421 mmol) in 2-methylpropan-2-ol (2 mL) at 20° C. The mixture was stirred for 15 minutes at 20° C. Then sodium periodate (43.9 g, 205 mmol) was added in portions at 0° C. The mixture was stirred for 2 hours at 20° C. Two additional reactions of the same type were run on 14.5 g and 5 g scales as described above. These three reaction mixtures were combined, extracted with ethyl acetate (400 mL), and filtered. The filtrate was quenched with saturated aqueous Na₂S₂O₃ solution (600 mL), and the mixture was extracted with ethyl acetate (3×200 mL). The combined organic phases were washed with brine (300 mL), dried over anhydrous sodium sulfate, filtered and concentrated under reduced pressure. The residue was purified by column chromatography on silica gel eluted with petroleum ether to give the title compound (19 g, 70.4 mmol, yield 57.2%, purity 95%). ¹H NMR (400 MHz, CDCl₃) δ ppm 9.79 (t, J=2.9 Hz, 1H), 3.41 (s, 2H), 2.39 (d, J=2.9 Hz, 2H), 1.71-1.66 (m, 6H), 1.52-1.41 (m, 2H), 0.88 (s, 9H), 0.01 (s, 6H).

### Example 6E: 5-[(3S)-5-fluoro-7-hydroxy-3-({2-[1-(hydroxymethyl)cyclopentyl]ethyl}amino)-3,4-dihydro-2H-1-benzothiopyran-6-yl]-1λ6,2,5-thiadiazolidine-1,1,3-trione

To a 4 mL vial were combined the product of Example 3P (60 mg, 0.162 mmol) and triethylamine (67.8 μL, 0.487 mmol, 3 equivalents) in 3:2 v/v ethanol/dichloromethane (1.6 mL) to give a suspension. The product of Example 6D (83.2 mg, 0.324 mmol, 2.0 equivalents) was added and the reaction mixture was stirred at room temperature for 2 hours. Sodium borohydride (24.5 mg, 0.649 mmol, 4.0 equivalents) was added in one portion, and the resultant mixture was stirred for 30 minutes at room temperature. The reaction mixture was cooled to 0° C. in an ice bath, aqueous hydrochloric acid (2.0 M, 1.6 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (12.8 mg, 17.2% yield). ¹H NMR (500 MHz, DMSO-d₆:D₂O=9:1 (v/v)) δ ppm 6.53 (d, J=1.4 Hz, 1H), 4.01 (s, 2H), 3.77-3.71 (m, 1H), 3.37-3.30 (m, 1H), 3.22 (s, 2H), 3.17-2.99 (m, 4H), 2.78-2.69 (m, 1H), 1.76-1.64 (m, 2H), 1.64-1.53 (m, 4H), 1.53-1.45 (m, 2H), 1.37-1.28 (m, 2H); MS (APCI⁺) m/z 460.4 [M+H]⁺.

### Example 7: 5-{(3S)-5-fluoro-7-hydroxy-3-[(4-hydroxy-3,3-dimethylbutyl)amino]-3,4-dihydro-2H-1-benzothiopyran-6-yl}-1λ⁶,2,5-thiadiazolidine-1,1,3-trione (Compound 106)

### Example 7A: methyl 2,2-dimethylpent-4-enoate

The title compound was synthesized from methyl isobutyrate using the methodology described for Example 6A in 71.8% yield. ¹H NMR (400 MHz CDCl₃) δ ppm 5.57-5.89 (m, 1H), 4.91-5.16 (m, 2H), 3.56 (s, 3H), 2.27 (m, 2H), 1.17 (s, 6H).

### Example 7B: 2,2-dimethylpent-4-en-1-ol

The title compound was synthesized from the product of Example 7A using the methodology described for Example 6B in 95% yield. ¹H NMR (400 MHz, CDCl₃) δ ppm 5.65-5.93 (m, 1H), 4.89-5.11 (m, 2H), 3.19-3.36 (m, 2H), 3.28 (s, 2H), 1.95 (d, J=7.63 Hz, 2H), 0.82 (s, 6H).

### Example 7C: tert-butyl[(2,2-dimethylpent-4-en-1-yl)oxy]dimethylsilane

The title compound was synthesized from the product of Example 7B using the procedure described for Example 6C in 74% yield. ¹H NMR (400 MHz, CDCl₃) δ ppm 5.63-6.07 (m, 1H), 4.86-5.21 (m, 2H), 3.21 (s, 2H), 2.00 (d, J=7.50 Hz, 2H), 0.91 (s, 9H), 0.84 (s, 6H), 0.01 (s, 6H).

### Example 7D: 4-{[tert-butyl(dimethyl)silyl]oxy}-3,3-dimethylbutanal

The title compound was synthesized from the product of Example 7C using the procedure described for Example 6D in 66% yield. ¹H NMR (400 MHz, CDCl₃) δ ppm 9.84 (t, J=3.q Hz, 1H) 3.35 (s, 2H) 2.28 (d, J=3.1 Hz, 2H) 1.02 (s, 6H) 0.89 (s, 9H) 0.04 (s, 6H), MS (ESI⁺) m/z 231 [M+H]⁺.

### Example 7E: 5-{(3S)-5-fluoro-7-hydroxy-3-[(4-hydroxy-3,3-dimethylbutyl)amino]-3,4-dihydro-2H-1-benzothiopyran-6-yl}-1λ⁶,2,5-thiadiazolidine-1,1,3-trione

In a 4 mL vial were combined the product of Example 3P (60 mg, 0.162 mmol) and triethylamine (67.8 μL, 0.487 mmol, 3 equivalents) in 3:2 v/v ethanol/dichloromethane (1.6 mL) to give a suspension. 4-{[tert-Butyl(dimethyl)silyl]oxy}-3,3-dimethylbutanal (Example 7D, 74.8 mg, 0.324 mmol, 2.0 equivalents) was added and the reaction mixture was stirred at room temperature for 2 hours. Sodium borohydride (24.5 mg, 0.649 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.6 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (10.3 mg, 14.4% yield). ¹H NMR (600 MHz, DMSO-d₆:D₂O=9:1 (v/v)) δ ppm 6.43 (d, J=1.3 Hz, 1H), 3.94 (d, J=1.0 Hz, 2H), 3.69-3.65 (m, 1H), 3.34-3.27 (m, 1H), 3.19-3.11 (m, 1H), 3.08 (s, 2H), 2.95 (dd, J=16.5, 4.6 Hz, 1H), 2.90 (dd, J=12.7, 8.9 Hz, 1H), 2.85-2.77 (m, 1H), 2.76-2.68 (m, 1H), 1.46-1.35 (m, 2H), 0.80 (d, J=1.8 Hz, 6H); MS (APCI⁺) m/z 434.3 [M+H]⁺.

### Example 8: 5-[(3S)-5-fluoro-7-hydroxy-3-(propylamino)-3,4-dihydro-2H-1-benzothiopyran-6-yl]-1λ⁶,2,5-thiadiazolidine-1,1,3-trione (Compound 107)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Propionaldehyde (23.6 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the reaction mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (3.3 mg, 6.5% yield). ¹H NMR (400 MHz, DMSO-d₆:D₂O=9:1 (v/v)) δ ppm 6.50 (d, J=1.5 Hz, 1H), 3.97 (s, 2H), 3.79-3.73 (m, 1H), 3.31 (d, J=12.8 Hz, 1H), 3.18-2.92 (m, 4H), 2.73 (dd, J=16.7, 8.2 Hz, 1H), 1.70-1.56 (m, 2H), 0.94 (t, J=7.4 Hz, 3H); MS (APCI⁺) m/z 376.3 [M+H]⁺.

### Example 9: 5-{(3S)-5-fluoro-7-hydroxy-3-[(3-methylpentyl)amino]-3,4-dihydro-2H-1-benzothiopyran-6-yl}-1λ⁶,2,5-thiadiazolidine-1,1,3-trione (Compound 108)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Methylpentanal (40.6 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (5.5 mg, 9.7% yield). ¹H NMR (400 MHz, DMSO-d₆:D₂O=9:1 (v/v)) δ ppm 6.50 (d, J=1.5 Hz, 1H), 3.97 (s, 2H), 3.81-3.73 (m, 1H), 3.36-3.28 (m, 1H), 3.18-2.99 (m, 4H), 2.73 (dd, J=16.8, 8.3 Hz, 1H), 1.68-1.57 (m, 1H), 1.49-1.41 (m, 2H), 1.36-1.26 (m, 1H), 1.25-1.10 (m, 1H), 0.92-0.82 (m, 6H); MS (APCI⁺) m/z 418.3 [M+H]⁺.

### Example 10: 5-[(3S)-5-fluoro-7-hydroxy-3-{[2-(oxan-4-yl)ethyl]amino}-3,4-dihydro-2H-1-benzothiopyran-6-yl]-1λ⁶,2,5-thiadiazolidine-1,1,3-trione (Compound 109)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-(Tetrahydro-2H-pyran-4-yl)acetaldehyde (52.0 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (6.5 mg, 10.8% yield). ¹H NMR (400 MHz, DMSO-d₆:D₂O=9:1 (v/v)) δ ppm 6.48 (d, J=1.5 Hz, 1H), 3.96 (s, 2H), 3.87-3.79 (m, 2H), 3.62-3.53 (m, 1H), 3.34-3.22 (m, 3H), 3.12-2.89 (m, 4H), 2.71-2.60 (m, 1H), 1.62-1.44 (m, 5H), 1.24-1.10 (m, 2H); MS (APCI⁺) m/z 446.4 [M+H]⁺.

### Example 11: 5-[(3S)-5-fluoro-7-hydroxy-3-{[(oxan-4-yl)methyl]amino}-3,4-dihydro-2H-1-benzothiopyran-6-yl]-1λ⁶,2,5-thiadiazolidine-1,1,3-trione (Compound 110)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Tetrahydro-2H-pyran-4-carbaldehyde (46.3 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (8.5 mg, 14.6% yield). ¹H NMR (400 MHz, DMSO-d₆:D₂O=9:1 (v/v)) δ ppm 6.50 (d, J=1.6 Hz, 1H), 3.97 (s, 2H), 3.91-3.82 (m, 2H), 3.75-3.70 (m, 1H), 3.36-3.23 (m, 3H), 3.21-3.07 (m, 2H), 2.98 (qd, J=12.4, 7.0 Hz, 2H), 2.72 (dd, J=16.5, 9.0 Hz, 1H), 1.97-1.86 (m, 1H), 1.72-1.63 (m, 2H), 1.33-1.18 (m, 2H); MS (APCI⁺) m/z 432.3 [M+H]⁺.

### Example 12: 5-{(3S)-3-[(cyclopropylmethyl)amino]-5-fluoro-7-hydroxy-3,4-dihydro-2H-1-benzothiopyran-6-yl}-1λ⁶,2,5-thiadiazolidine-1,1,3-trione (Compound 111)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Cyclopropanecarbaldehyde (28.4 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (9.8 mg, 18.8% yield). ¹H NMR (400 MHz, DMSO-d₆:D₂O=9:1 (v/v)) δ ppm 6.50 (d, J=1.5 Hz, 1H), 3.97 (s, 2H), 3.85-3.73 (m, 1H), 3.35-3.26 (m, 1H), 3.19-3.06 (m, 2H), 3.05-2.91 (m, 2H), 2.74 (dd, J=16.7, 8.6 Hz, 1H), 1.12-0.98 (m, 1H), 0.68-0.56 (m, 2H), 0.43-0.32 (m, 2H); MS (APCI⁺) m/z 388.3 [M+H]⁺.

### Example 13: 5-[(3S)-3-(butylamino)-5-fluoro-7-hydroxy-3,4-dihydro-2H-1-benzothiopyran-6-yl]-1λ⁶,2,5-thiadiazolidine-1,1,3-trione (Compound 112)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Butyraldehyde (29.3 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (10.0 mg, 19% yield). ¹H NMR (400 MHz, DMSO-d₆:D₂O=9:1 (v/v)) δ ppm 6.50 (d, J=1.5 Hz, 1H), 3.97 (s, 2H), 3.81-3.72 (m, 1H), 3.36-3.27 (m, 1H), 3.20-2.96 (m, 4H), 2.74 (dd, J=16.7, 8.3 Hz, 1H), 1.66-1.54 (m, 2H), 1.36 (h, J=7.4 Hz, 2H), 0.92 (t, J=7.4 Hz, 3H); MS (APCI⁺) m/z 390.3 [M+H]⁺.

### Example 14: 5-{(3S)-5-fluoro-7-hydroxy-3-[(2-methylpropyl)amino]-3,4-dihydro-2H-1-benzothiopyran-6-yl}-1λ⁶,2,5-thiadiazolidine-1,1,3-trione (Compound 113)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Isobutyraldehyde (29.5 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (14.1 mg, 26.8% yield). ¹H NMR (400 MHz, DMSO-d₆:D₂O=9:1 (v/v)) δ ppm 6.50 (d, J=1.5 Hz, 1H), 3.97 (s, 2H), 3.78-3.71 (m, 1H), 3.36-3.27 (m, 1H), 3.23-3.08 (m, 2H), 3.00-2.86 (m, 2H), 2.73 (dd, J=16.6, 9.3 Hz, 1H), 2.08-1.89 (m, 1H), 0.98 (d, J=6.7 Hz, 6H); MS (APCI⁺) m/z 390.3 [M+H]⁺.

### Example 15: 5-[(3S)-5-fluoro-7-hydroxy-3-{[2-(oxolan-3-yl)ethyl]amino}-3,4-dihydro-2H-1-benzothiopyran-6-yl]-1λ⁶,2,5-thiadiazolidine-1,1,3-trione (Compound 114)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-(Tetrahydrofuran-3-yl)acetaldehyde (46.3 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (16.1 mg, 27.6% yield). ¹H NMR (400 MHz, DMSO-d₆:D₂O=9:1 (v/v)) δ ppm 6.51 (d, J=1.5 Hz, 1H), 3.97 (s, 2H), 3.83-3.58 (m, 4H), 3.36-3.22 (m, 2H), 3.20-2.97 (m, 4H), 2.74 (dd, J=16.7, 8.2 Hz, 1H), 2.28-2.12 (m, 1H), 2.09-1.96 (m, 1H), 1.77-1.60 (m, 2H), 1.56-1.42 (m, 1H); MS (APCI⁺) m/z 432.3 [M+H]⁺.

### Example 16: 5-{(3S)-3-[(2,3-dimethylbutyl)amino]-5-fluoro-7-hydroxy-3,4-dihydro-2H-1-benzothiopyran-6-yl}-1λ⁶,2,5-thiadiazolidine-1,1,3-trione (Compound 115)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2,3-Dimethylbutanal (40.6 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (16.1 mg, 28.6% yield). ¹H NMR (400 MHz, DMSO-d₆:D₂O=9:1 (v/v)) δ ppm 6.50 (d, J=1.5 Hz, 1H), 3.97 (s, 2H), 3.76-3.71 (m, 1H), 3.37-3.26 (m, 1H), 3.23-2.97 (m, 3H), 2.93-2.67 (m, 2H), 1.78-1.63 (m, 2H), 0.90 (dd, J=6.6, 2.4 Hz, 6H), 0.83 (dd, J=6.7, 2.1 Hz, 3H); MS (APCI⁺) m/z 418.3 [M+H]⁺.

### Example 17: 5-{(3S)-5-fluoro-7-hydroxy-3-[(propan-2-yl)amino]-3,4-dihydro-2H-1-benzothiopyran-6-yl}-1λ⁶,2,5-thiadiazolidine-1,1,3-trione (Compound 116)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Acetone (23.5 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (3.2 mg, 6.3% yield). ¹H NMR (600 MHz, DMSO-d₆:D₂O=9:1 (v/v)) δ ppm 6.56 (d, J=1.3 Hz, 1H), 4.04 (s, 2H), 3.79-3.73 (m, 1H), 3.59-3.49 (m, 1H), 3.34-3.29 (m, 1H), 3.19-3.07 (m, 2H), 2.70 (dd, J=16.2, 9.0 Hz, 1H), 1.32-1.27 (m, 6H); MS (APCI⁺) m/z 376.3 [M+H]⁺.

### Example 18: 5-[(3S)-3-{[2-(3,3-difluorocyclobutyl)ethyl]amino}-5-fluoro-7-hydroxy-3,4-dihydro-2H-1-benzothiopyran-6-yl]-1λ⁶,2,5-thiadiazolidine-1,1,3-trione (Compound 117)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-(3,3-Difluorocyclobutyl)acetaldehyde (54.4 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (6.4 mg, 10.6% yield). ¹H NMR (600 MHz, DMSO-d₆:D₂O=9:1 (v/v)) δ ppm 6.53 (d, J=1.4 Hz, 1H), 4.01 (s, 2H), 3.75-3.60 (m, 1H), 3.31 (s, 1H), 3.10 (q, J=13.7 Hz, 2H), 2.97 (d, J=25.3 Hz, 2H), 2.78-2.67 (m, 3H), 2.40-2.14 (m, 3H), 1.82 (q, J=7.7 Hz, 2H); MS (APCI⁺) m/z 452.3 [M+H]⁺.

### Example 19: 5-{(3S)-3-[(2-cyclohexylethyl)amino]-5-fluoro-7-hydroxy-3,4-dihydro-2H-1-benzothiopyran-6-yl}-1λ⁶,2,5-thiadiazolidine-1,1,3-trione (Compound 118)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-Cyclohexylacetaldehyde (48.7 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (11.2 mg, 18.6% yield). ¹H NMR (400 MHz, DMSO-d₆:D₂O=9:1 (v/v)) δ ppm 6.47 (d, J=1.5 Hz, 1H), 3.95 (s, 2H), 3.61-3.55 (m, 1H), 3.24 (d, J=12.8 Hz, 1H), 3.10-2.88 (m, 4H), 2.63 (dd, J=16.7, 8.4 Hz, 1H), 1.65 (td, J=17.8, 15.0, 4.9 Hz, 5H), 1.50-1.40 (m, 2H), 1.36-1.04 (m, 4H), 0.96-0.82 (m, 2H); MS (APCI⁺) m/z 444.3 [M+H]⁺.

### Example 20: 5-{(3S)-3-[(3-ethylpentyl)amino]-5-fluoro-7-hydroxy-3,4-dihydro-2H-1-benzothiopyran-6-yl}-1λ⁶,2,5-thiadiazolidine-1,1,3-trione (Compound 119)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Ethylpentanal (46.1 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to afford the title compound (11.7 mg, 20.1% yield). ¹H NMR (400 MHz, DMSO-d₆:D₂O=9:1 (v/v)) δ ppm 6.47 (d, J=1.5 Hz, 1H), 3.96 (s, 2H), 3.57-3.50 (m, 1H), 3.23 (dd, J=12.0, 2.9 Hz, 1H), 3.08-2.79 (m, 4H), 2.62 (dd, J=16.6, 8.4 Hz, 1H), 1.54-1.44 (m, 2H), 1.32-1.22 (m, 5H), 0.82 (t, J=6.9 Hz, 6H); MS (APCI⁺) m/z 432.3 [M+H]⁺.

### Example 21: 5-{(3S)-3-[(2-cyclopentylethyl)amino]-5-fluoro-7-hydroxy-3,4-dihydro-2H-1-benzothiopyran-6-yl}-1λ⁶,2,5-thiadiazolidine-1,1,3-trione (Compound 120)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-Cyclopentylacetaldehyde (45.5 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (18.6 mg, 32% yield). ¹H NMR (600 MHz, DMSO-d₆:D₂O=9:1 (v/v)) δ ppm 6.57 (d, J=1.5 Hz, 1H), 4.04 (s, 2H), 3.88-3.82 (m, 1H), 3.41-3.35 (m, 1H), 3.23-3.04 (m, 4H), 2.80 (dd, J=16.7, 8.2 Hz, 1H), 1.93-1.79 (m, 3H), 1.74-1.64 (m, 4H), 1.63-1.48 (m, 2H), 1.22-1.13 (m, 2H); MS (APCI⁺) m/z 430.3 [M+H]⁺.

### Example 22: 5-[(3S)-3-(benzylamino)-5-fluoro-7-hydroxy-3,4-dihydro-2H-1-benzothiopyran-6-yl]-1λ⁶,2,5-thiadiazolidine-1,1,3-trione (Compound 121)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Benzaldehyde (43.0 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (21.2 mg, 37.1% yield). ¹H NMR (500 MHz, DMSO-d₆:D₂O=9:1 (v/v)) δ ppm 7.55-7.38 (m, 5H), 6.53 (s, 1H), 4.20-4.07 (m, 2H), 4.03 (s, 2H), 3.56-3.37 (m, 1H), 3.32 (d, J=12.5 Hz, 1H), 3.15-3.03 (m, 2H), 2.71-2.62 (m, 1H); MS (APCI⁺) m/z 424.3 [M+H]⁺.

### Example 23: 5-{(3S)-5-fluoro-7-hydroxy-3-[(3-methylbut-2-en-1-yl)amino]-3,4-dihydro-2H-1-benzothiopyran-6-yl}-1λ⁶,2,5-thiadiazolidine-1,1,3-trione (Compound 122)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Methoxy-3-methylbutanal (47.1 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (13.2 mg, 24.3% yield). ¹H NMR (400 MHz, DMSO-d₆:D₂O=9:1 (v/v)) δ ppm 6.49 (d, J=1.6 Hz, 1H), 5.25 (tt, J=7.3, 1.5 Hz, 1H), 3.97 (s, 2H), 3.68-3.57 (m, 3H), 3.35-3.27 (m, 1H), 3.15-3.01 (m, 2H), 2.71 (dd, J=16.7, 8.3 Hz, 1H), 1.73 (dd, J=19.1, 1.4 Hz, 6H); MS (APCI⁺) m/z 402.3 [M+H]⁺.

### Example 24: 5-{(3S)-5-fluoro-7-hydroxy-3-[(2-phenylethyl)amino]-3,4-dihydro-2H-1-benzothiopyran-6-yl}-1λ⁶,2,5-thiadiazolidine-1,1,3-trione (Compound 123)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Phenylacetaldehyde (48.7 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 3.24 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (9.7 mg, 16.4% yield). ¹H NMR (500 MHz, DMSO-d₆:D₂O=9:1 (v/v)) δ ppm 7.36-7.21 (m, 5H), 6.47 (d, J=1.5 Hz, 1H), 3.95 (s, 2H), 3.84-3.73 (m, 1H), 3.32-3.17 (m, 3H), 3.13-3.05 (m, 2H), 2.91 (t, J=7.9 Hz, 2H), 2.72 (dd, J=16.7, 8.1 Hz, 1H); MS (APCI⁺) m/z 438.3 [M+H]⁺.

### Example 25: 5-{(3S)-3-[(3,3-dimethylpentyl)amino]-5-fluoro-7-hydroxy-3,4-dihydro-2H-1-benzothiopyran-6-yl}-1λ⁶,2,5-thiadiazolidine-1,1,3-trione (Compound 124)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3,3-Dimethylpentanal (46.3 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (21.2 mg, 36.4% yield). ¹H NMR (400 MHz, DMSO-d₆:D₂O=9:1 (v/v)) δ ppm 6.47 (d, J=1.5 Hz, 1H), 3.95 (s, 2H), 3.58-3.54 (m, 1H), 3.29-3.21 (m, 1H), 3.10-2.78 (m, 4H), 2.62 (dd, J=16.7, 8.4 Hz, 1H), 1.52-1.38 (m, 2H), 1.21 (q, J=7.5 Hz, 2H), 0.86-0.72 (m, 9H); MS (APCI⁺) m/z 432.4 [M+H]⁺.

### Example 26: 5-[(3S)-5-fluoro-3-{[(1-fluorocyclopropyl)methyl]amino}-7-hydroxy-3,4-dihydro-2H-1-benzothiopyran-6-yl]-1λ⁶,2,5-thiadiazolidine-1,1,3-trione (Compound 125)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 1-Fluorocyclopropane-1-carbaldehyde (35.7 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (20.1 mg, 36.6% yield). ¹H NMR (500 MHz, DMSO-d₆:D₂O=9:1 (v/v)) δ ppm 6.49 (d, J=1.4 Hz, 1H), 4.00 (s, 2H), 3.43-3.34 (m, 1H), 3.26-3.14 (m, 3H), 3.06-2.90 (m, 2H), 2.56-2.50 (m, 1H), 1.10-1.02 (m, 2H), 0.85-0.73 (m, 2H); MS (APCI⁺) m/z 406.2 [M+H]⁺.

### Example 27: 5-[(3S)-3-{[(2,2-difluorocyclopropyl)methyl]amino}-5-fluoro-7-hydroxy-3,4-dihydro-2H-1-benzothiopyran-6-yl]-1λ⁶,2,5-thiadiazolidine-1,1,3-trione (Compound 126)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2,2-Difluorocyclopropane-1-carbaldehyde (43.0 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (18.4 mg, 32.2% yield). ¹H NMR (400 MHz, DMSO-d₆:D₂O=9:1 (v/v)) δ ppm 6.45 (s, 1H), 3.95 (s, 2H), 3.43-3.38 (m, 1H), 3.22-3.13 (m, 1H), 2.98 (d, J=20.8 Hz, 4H), 2.61-2.53 (m, 1H), 1.97-1.83 (m, 1H), 1.67-1.60 (m, 1H), 1.40-1.25 (m, 1H); MS (APCI⁺) m/z 424.3 [M+H]⁺.

### Example 28: 5-[(3S)-5-fluoro-7-hydroxy-3-{[(3-methyloxetan-3-yl)methyl]amino}-3,4-dihydro-2H-1-benzothiopyran-6-yl]-1λ⁶,2,5-thiadiazolidine-1,1,3-trione (Compound 127)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Methyloxetane-3-carbaldehyde (40.6 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A) to afford the title compound (18.1 mg, 32.0% yield). ¹H NMR (400 MHz, DMSO-d₆:D₂O=9:1 (v/v)) δ ppm 6.49 (d, J=1.5 Hz, 1H), 4.42 (dd, J=5.9, 2.2 Hz, 2H), 4.29-4.23 (m, 2H), 4.00 (s, 2H), 3.47-3.15 (m, 2H), 3.10-2.92 (m, 4H), 2.57-2.50 (m, 1H), 1.31 (s, 3H); MS (APCI⁺) m/z 418.3 [M+H]⁺.

### Example 29: 5-[(3S)-5-fluoro-3-({[1-(fluoromethyl)cyclopropyl]methyl}amino)-7-hydroxy-3,4-dihydro-2H-1-benzothiopyran-6-yl]-1λ⁶,2,5-thiadiazolidine-1,1,3-trione (Compound 128)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 1-(Fluoromethyl)cyclopropane-1-carbaldehyde (41.4 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (12.8 mg, 22.6% yield). ¹H NMR (400 MHz, DMSO-d₆:D₂O=9:1 (v/v)) δ ppm 6.45 (d, J=1.5 Hz, 1H), 4.42-4.37 (m, 1H), 4.30-4.25 (m, 1H), 3.95 (s, 2H), 3.49-3.33 (m, 1H), 3.23-3.15 (m, 1H), 3.04-2.91 (m, 4H), 2.63-2.53 (m, 1H), 0.62 (s, 4H); MS (APCI⁺) m/z 420.3 [M+H]⁺.

### Example 30: 5-{(3S)-3-[(4,4-difluoropentyl)amino]-5-fluoro-7-hydroxy-3,4-dihydro-2H-1-benzothiopyran-6-yl}-1λ⁶,2,5-thiadiazolidine-1,1,3-trione (Compound 129)

A 1 dram vial was charged with 5-[(3S)-3-amino-5-fluoro-7-hydroxy-3,4-dihydro-2H-1-benzothiopyran-6-yl]-1λ⁶,2,5-thiadiazolidine-1,1,3-trione hydrochloride (33 mg, 0.089 mmol, Example 3P), dichloromethane (535 μL), ethanol (357 μL), and triethylamine (37.3 μL, 0.268 mmol). Subsequently, 4,4-difluoropentanal (32.7 mg, 0.268 mmol) was added and the solution was stirred for 2 hours before the addition of sodium borohydride (13.50 mg, 0.357 mmol) in one portion. After 30 minutes, the reaction was quenched with aqueous 3 M HCl (595 μL, 1.785 mmol), and the mixture was partially concentrated under vacuum. The solution was diluted with aqueous ammonium bicarbonate (0.025 M in water, acidified to pH 7 by addition of dry ice) and loaded onto a 30 g Biotage® Sfar C18 column, where it was purified by a 10-100% gradient of methanol in 0.025 M ammonium bicarbonate in water (acidified to pH 7 by addition of dry ice) to yield the title compound (17.8 mg, 0.041 mmol, 45.4% yield). ¹H NMR (499.6 MHz, DMSO-d₆) δ ppm 9.51 (s, 1H), 8.59 (s, 1H), 6.46 (s, 1H), 3.91 (s, 2H), 3.26 (m, 2H), 3.17-3.04 (m, 4H), 2.71-2.63 (m, 1H), 2.02-1.91 (m, 2H), 1.77-1.70 (m, 2H); MS (APCI⁺) m/z 440 [M+H]⁺.

### Example 31: 5-{(3S)-5-fluoro-7-hydroxy-3-[(3-methoxypropyl)amino]-3,4-dihydro-2H-1-benzothiopyran-6-yl}-1λ⁶,2,5-thiadiazolidine-1,1,3-trione (Compound 130)

A 1 dram vial was charged with 5-[(3S)-3-amino-5-fluoro-7-hydroxy-3,4-dihydro-2H-1-benzothiopyran-6-yl]-1λ⁶,2,5-thiadiazolidine-1,1,3-trione hydrochloride (33 mg, 0.089 mmol, Example 3P), dichloromethane (535 μL), ethanol (357 μL), and triethylamine (37.3 μL, 0.268 mmol). Subsequently, 3-methoxypropanal (23.12 μL, 0.268 mmol) was added, and the solution was stirred for 2 hours before the addition of sodium borohydride (13.50 mg, 0.357 mmol) in one portion. After 30 minutes, the reaction was quenched with aqueous 3 M ammonium chloride (54.2 μL, 1.785 mmol) and partially concentrated under a stream of nitrogen. The solution was diluted with aqueous ammonium bicarbonate (0.025 M in water, acidified to pH 7 by addition of dry ice) and loaded onto a 30 g Biotage® Sfar C18 column, where it was purified by a 10-100% gradient of methanol in 0.025 ammonium bicarbonate in water (acidified to pH 7 by addition of dry ice) to yield the title compound (10.2 mg, 0.025 mmol, 28.2% yield). ¹H NMR (600.4 MHz, DMSO-d₆) δ ppm 9.52 (s, 1), 8.47 (s, 1H), 6.47 (s, 1H), 3.91 (ABq, J=13.2 Hz, 2H), 3.40 (t, J=6.0 Hz, 2H), 3.28 (m, 2H), 3.24 (s, 1H), 3.11-3.05 (m. 4H), 2.70 (dd, J=16.8, 8.0 Hz, 1H), 1.83 (p, J=6.7 Hz, 2H); (APCI⁺) m/z 406 [M+H]⁺.

### Example 32: 5-[(3S)-5-fluoro-7-hydroxy-3-{[3-(1H-pyrazol-1-yl)propyl]amino}-3,4-dihydro-2H-1-benzothiopyran-6-yl]-1λ⁶,2,5-thiadiazolidine-1,1,3-trione (Compound 131)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-(1H-Pyrazol-1-yl)propanal (33.6 mg, 0.27 mmol, 2.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (29.1 mg, 48.8% yield). ¹H NMR (500 MHz, DMSO-d₆:D₂O=9:1 (v/v)) δ ppm 7.73-7.68 (m, 1H), 7.49-7.41 (m, 1H), 6.46 (d, J=1.4 Hz, 1H), 6.26-6.23 (m, 1H), 4.19 (t, J=6.8 Hz, 2H), 3.95 (s, 2H), 3.56-3.50 (m, 1H), 3.23-3.16 (m, 1H), 3.03-2.92 (m, 2H), 2.94-2.82 (m, 2H), 2.61 (dd, J=16.7, 8.1 Hz, 1H), 2.04 (p, J=7.1 Hz, 2H); MS (APCI⁺) m/z 442.3 [M+H]⁺.

### Example 33: 5-[(3S)-5-fluoro-7-hydroxy-3-{[2-(1-methylcyclopropyl)ethyl]amino}-3,4-dihydro-2H-1-benzothiopyran-6-yl]-1λ⁶,2,5-thiadiazolidine-1,1,3-trione (Compound 132)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-(1-Methylcyclopropyl)acetaldehyde (39.8 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (15.5 mg, 27.6% yield). ¹H NMR (500 MHz, DMSO-d₆:D₂O=9:1 (v/v)) δ ppm 6.54 (d, J=1.4 Hz, 1H), 4.01 (s, 2H), 3.81-3.78 (m, 1H), 3.40-3.33 (m, 1H), 3.26-3.03 (m, 4H), 2.78 (dd, J=16.8, 8.1 Hz, 1H), 1.65-1.53 (m, 2H), 1.07 (s, 3H), 0.43-0.36 (m, 2H), 0.36-0.28 (m, 2H); MS (APCI⁺) m/z 416.3 [M+H]⁺.

### Example 34: 5-{(3S)-3-[(2-cyclopropylpropyl)amino]-5-fluoro-7-hydroxy-3,4-dihydro-2H-1-benzothiopyran-6-yl}-1λ⁶,2,5-thiadiazolidine-1,1,3-trione (Compound 133)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-Cyclopropylpropanal (39.8 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A) to afford the title compound (12.0 mg, 21.4% yield). ¹H NMR (600 MHz, DMSO-d₆:D₂O=9:1 (v/v)) δ ppm 6.39 (s, 1H), 3.86 (d, J=1.2 Hz, 2H), 3.73-3.64 (m, 1H), 3.23-3.16 (m, 1H), 3.11-2.89 (m, 4H), 2.62 (dd, J=16.4, 9.5 Hz, 1H), 1.06-0.98 (m, 1H), 0.91 (dd, J=6.8, 1.2 Hz, 3H), 0.52-0.46 (m, 1H), 0.40-0.29 (m, 2H), 0.17-0.12 (m, 1H), −0.01 (dtd, J=10.8, 6.2, 5.6, 3.1 Hz, 1H); MS (APCI⁺) m/z 416.3 [M+H]⁺.

### Example 35: 5-{(3S)-5-fluoro-7-hydroxy-3-[(3-phenylbutyl)amino]-3,4-dihydro-2H-1-benzothiopyran-6-yl}-1λ⁶,2,5-thiadiazolidine-1,1,3-trione (Compound 134)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Phenylbutanal (60.1 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to afford the title compound (2.7 mg, 4.2% yield). ¹H NMR (400 MHz, DMSO-d₆:D₂O=9:1 (v/v)) δ ppm 7.34 (t, J=7.5 Hz, 2H), 7.30-7.19 (m, 3H), 6.49 (s, 1H), 3.96 (s, 2H), 3.87-3.74 (m, 1H), 3.27 (s, 1H), 3.06 (d, J=14.2 Hz, 3H), 2.90-2.63 (m, 3H), 1.89 (q, J=7.9 Hz, 2H), 1.24 (d, J=6.9 Hz, 3H).

### Example 36: 5-[(3S)-5-fluoro-7-hydroxy-3-{[(3-phenylcyclobutyl)methyl]amino}-3,4-dihydro-2H-1-benzothiopyran-6-yl]-1λ⁶,2,5-thiadiazolidine-1,1,3-trione (Compound 135)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Phenylcyclobutane-1-carbaldehyde (65.0 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. The mixture was then partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (3.5 mg, 5.5% yield). ¹H NMR (400 MHz, DMSO-d₆:D₂O=9:1 (v/v)) δ ppm 7.38-7.16 (m, 5H), 6.50 (d, J=1.6 Hz, 1H), 3.98 (d, J=1.5 Hz, 2H), 3.69-3.59 (m, 1H), 3.44-3.26 (m, 2H), 3.07 (s, 3H), 2.71 (dd, J=15.0, 10.1 Hz, 1H), 2.53-2.44 (m, 3H), 2.36-2.22 (m, 1H), 1.92-1.81 (m, 2H); MS (APCI⁺) m/z 478.4 [M+H]⁺.

### Example 37: 5-{(3S)-5-fluoro-7-hydroxy-3-[(3-phenylpropyl)amino]-3,4-dihydro-2H-1-benzothiopyran-6-yl}-1λ⁶,2,5-thiadiazolidine-1,1,3-trione (Compound 136)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-Phenylpropanal (54.4 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. The mixture was then partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (13.4 mg, 22% yield). ¹H NMR (400 MHz, DMSO-d₆:D₂O=9:1 (v/v)) δ ppm 7.37-7.15 (m, 5H), 6.50 (d, J=1.5 Hz, 1H), 3.97 (s, 2H), 3.33-3.25 (m, 1H), 3.15-3.01 (m, 4H), 2.71 (dt, J=26.9, 8.0 Hz, 3H), 1.98-1.86 (m, 2H); MS (APCI⁺) m/z 452.4 [M+H]⁺.

### Example 38: 5-[(3S)-3-{[3-(2,2-difluoroethoxy)propyl]amino}-5-fluoro-7-hydroxy-3,4-dihydro-2H-1-benzothiopyran-6-yl]-1λ⁶,2,5-thiadiazolidine-1,1,3-trione (Compound 137)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 3-(2,2-Difluoroethoxy)propanal (56.0 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. The mixture was then partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (15.6 mg, 25.4% yield). ¹H NMR (400 MHz, DMSO-d₆:D₂O=9:1 (v/v)) δ ppm 6.51 (d, J=1.5 Hz, 1H), 6.12 (tt, J=54.9, 3.6 Hz, 1H), 3.97 (s, 2H), 3.83-3.79 (m, 1H), 3.72 (s, 3H), 3.35-3.28 (m, 1H), 3.21-3.03 (m, 5H), 2.81-2.70 (m, 1H), 1.96-1.84 (m, 2H); MS (APCI⁺) m/z 456.3 [M+H]⁺.

### Example 39: 5-[(3S)-3-({[(1RS,5SR)-bicyclo[3.1.0]hexan-6-yl]methyl}amino)-5-fluoro-7-hydroxy-3,4-dihydro-2H-1-benzothiopyran-6-yl]-1λ⁶,2,5-thiadiazolidine-1,1,3-trione (Compound 138)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. Bicyclo[3.1.0]hexane-6-carbaldehyde (44.7 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (16.5 mg, 28.5% yield). ¹H NMR (400 MHz, DMSO-d₆:D₂O=9:1 (v/v)) δ ppm 6.50 (d, J=1.5 Hz, 1H), 3.98 (s, 2H), 3.80-3.74 (m, 1H), 3.34-3.05 (m, 3H), 3.02-2.87 (m, 2H), 2.74 (dd, J=16.7, 8.5 Hz, 1H), 1.79-1.48 (m, 5H), 1.37-1.23 (m, 2H), 1.16-0.99 (m, 1H), 0.90-0.80 (m, 1H); MS (APCI⁺) m/z 428.3 [M+H]⁺.

### Example 40: 5-[(3S)-5-fluoro-7-hydroxy-3-({[4-(trifluoromethyl)cyclohexyl]methyl}amino)-3,4-dihydro-2H-1-benzothiopyran-6-yl]-1λ⁶,2,5-thiadiazolidine-1,1,3-trione (Compound 139)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 4-(Trifluoromethyl)cyclohexane-1-carbaldehyde (73.1 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. The mixture was then partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (18.6 mg, 27.6% yield). ¹H NMR (400 MHz, DMSO-d₆:D₂O=9:1 (v/v)) δ ppm 6.50 (d, J=1.7 Hz, 1H), 3.98 (s, 2H), 3.37-3.25 (m, 1H), 3.23-3.00 (m, 3H), 2.97-2.86 (m, 1H), 2.78-2.66 (m, 1H), 2.40-2.15 (m, 1H), 2.03-1.81 (m, 2H), 1.77-1.42 (m, 5H), 1.33-1.19 (m, 1H), 1.06 (q, J=12.7 Hz, 1H); MS (APCI⁺) m/z 498.1 [M+H]⁺.

### Example 41: 5-[(3S)-5-fluoro-7-hydroxy-3-{[2-(2,6,6-trimethylcyclohex-1-en-1-yl)ethyl]amino}-3,4-dihydro-2H-1-benzothiopyran-6-yl]-1λ⁶,2,5-thiadiazolidine-1,1,3-trione (Compound 140)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. 2-(2,6,6-Trimethylcyclohex-1-en-1-yl)acetaldehyde (67.4 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (21.3 mg, 32.6% yield). ¹H NMR (400 MHz, DMSO-d₆:D₂O=9:1 (v/v)) δ ppm 6.52 (d, J=1.6 Hz, 1H), 3.97 (s, 2H), 3.86-3.81 (m, 1H), 3.32 (d, J=12.0 Hz, 1H), 3.19-3.06 (m, 2H), 3.02-2.90 (m, 2H), 2.81-2.69 (m, 1H), 2.35 (t, J=8.9 Hz, 2H), 1.90 (t, J=6.1 Hz, 2H), 1.63 (s, 3H), 1.55-1.51 (m, 2H), 1.44-1.36 (m, 2H), 0.99 (s, 6H); MS (APCI⁺) m/z 484.4 [M+H]⁺.

### Example 42: tert-butyl 4-({[(3S)-5-fluoro-7-hydroxy-6-(1,1,4-trioxo-1λ⁶,2,5-thiadiazolidin-2-yl)-3,4-dihydro-2H-1-benzothiopyran-3-yl]amino}methyl)piperidine-1-carboxylate (Compound 141)

In a 4 mL vial were combined the product of Example 3P (50 mg, 0.14 mmol) and triethylamine (57 μL, 0.41 mmol, 3 equivalents) in 3:2 v/v ethanol/dichloromethane (1.4 mL) to give a suspension. tert-Butyl 4-formylpiperidine-1-carboxylate (86.5 mg, 0.41 mmol, 3.0 equivalents) was added, and the reaction mixture was stirred at room temperature for 2 hours. Sodium borohydride (20.5 mg, 0.54 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A) to afford the title compound (23.5 mg, 32.8% yield). ¹H NMR (400 MHz, DMSO-d₆:D₂O=9:1 (v/v)) δ ppm 6.50 (s, 1H), 3.95 (d, J=18.0 Hz, 4H), 3.81-3.71 (m, 1H), 3.34-3.22 (m, 1H), 3.20-2.86 (m, 5H), 2.81-2.58 (m, 3H), 1.73 (d, J=12.9 Hz, 2H), 1.39 (d, J=4.0 Hz, 9H), 1.08 (d, J=12.3 Hz, 2H); MS (APCI⁺) m/z 531.4 [M+H]⁺.

### Biological Assays

### Abbreviations

DMEM for Dulbecco's Modified Eagle Medium; DMSO for dimethyl sulfoxide; DTT for dithiothreitol; EDTA for ethylenediaminetetraacetic acid; EGTA for ethylene glycol-bis(2-aminoethylether)-N,N,N′,N′-tetraacetic acid; HEPES for 4-(2-hydroxyethyl)piperazine-1-ethanesulfonic acid; IFNγ for interferon gamma; and Tween® 20 for polyethylene glycol sorbitan monolaurate.

### Example 43: Mobility Shift Assay (MSA) Used to Determine Potency of PTPN2 Inhibitors

Compound activity was determined using in house His tagged PTPN2 (TC45) protein (SEQ ID NO: 1) in an in vitro enzymatic reaction. The enzymatic assay used to determine activity was a mobility shift assay using a LabChip EZ Reader by Caliper Life Sciences. The enzymatic reaction was carried out in assay buffer (50 mM HEPES pH 7.5, 1 mM EGTA, 10 mM EDTA, 0.01% Tween® 20, and 2 mM DTT). The compounds were dispensed on a white 384 well ProxiPlate™ (PerkinElmer Catalog #6008289) plate using the Labcyte Echo at varying concentrations (12 point, 1:3 dilution). The enzyme (at 0.5 nM) was incubated with compound for 10 minutes at room temperature. Thereafter, the substrate (phosphorylated insulin receptor probe sequence: ((OG488)-(NH—CH₂—CH₂—O—CH₂—CH₂—O—CH₂—CO)-T-R-D-I—(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN2). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which were used to calculate % inhibition. The % inhibition was then used to calculate the IC₅₀ values.

### Example 44: Mobility Shift Assay (MSA) Used to Determine Potency of PTPN1 Inhibitors

Compound activity was determined using in house His tagged full-length PTPN1 protein (SEQ ID NO: 3) in an in vitro enzymatic reaction. The enzymatic assay used to determine activity is a mobility shift assay using a LabChip EZ Reader by Caliper Life Sciences. The enzymatic reaction was carried out in assay buffer (50 mM HEPES pH 7.5, 1 mM EGTA, 10 mM EDTA, 0.010% Tween® 20, and 2 mM DTT). The compounds were dispensed on a white 384 well ProxiPlate™ (PerkinElmer Cat #6008289) plate using a Labcyte Echo® liquid handler at varying concentrations (12 point, 1:3 dilution). The enzyme (at 0.5 nM) was incubated with compound for 10 minutes at room temperature. Thereafter, the substrate (phosphorylated insulin receptor probe sequence: ((OG488)-(NH—CH₂—CH₂—O—CH₂—CH₂—O—CH₂—CO)-T-R-D-I—(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN1). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which were used to calculate % inhibition. The % inhibition was then used to calculate the IC₅₀ values.

Table 1 below summarizes the IC₅₀ data obtained using the PTPN2 MSA assay and the PTPN1 MSA assay for exemplary compounds of the disclosure. In this table, “A” represents an IC₅₀ of less than 10 nM; “B” an IC₅₀ of between 10 nM and 100 nM; and “C” an IC₅₀ of greater than 100 nM to 100 nM.

TABLE 1
IC₅₀ values of exemplary compounds of the disclosure in the
PTPN2 and PTPN1 Mobility Shift Assays (MSA).

| Compound | PTPN2 | PTPN1 | Compound | PTPN2 | PTPN1 |
|---|---|---|---|---|---|
| No. | MSA IC₅₀ | MSA IC₅₀ | No. | MSA IC₅₀ | MSA IC₅₀ |
| 100 | A | A | 101 | | C |
| 102 | B | C | 103 | A | A |
| 104 | A | B | 105 | A | B |
| 106 | A | B | 107 | B | B |
| 108 | A | A | 109 | A | B |
| 110 | B | B | 111 | B | B |
| 112 | A | B | 113 | B | B |
| 114 | A | B | 115 | A | A |
| 116 | B | C | 117 | A | B |
| 118 | A | A | 119 | A | B |
| 120 | A | A | 121 | B | B |
| 122 | B | B | 123 | A | B |
| 124 | A | B | 125 | B | B |
| 126 | B | B | 127 | B | B |
| 128 | B | B | 129 | A | A |
| 130 | B | B | 131 | B | C |
| 132 | A | B | 133 | A | A |
| 134 | A | B | 135 | A | B |
| 136 | B | B | 137 | B | B |
| 138 | B | B | 139 | A | B |
| 140 | B | B | 141 | B | B |

### Example 45: B16F10 (Murine Melanoma Cells) Phospho-STAT1 HTRF Proximal Pharmacodynamic (PD) Assay

B16F10 cells were grown and maintained in high glucose DMEM (Gibco, Catalog #11965-092, Dun Laoghaire Co Dublin) supplemented with 10% fetal bovine serum (Gibco, Catalog #10082-139, Dun Laoghaire Co Dublin). The cells were pelleted and resuspended in high glucose DMEM without phenol red (Gibco, Catalog #11054-020, Dun Laoghaire Co Dublin), supplemented with 10% fetal bovine serum and plated in a 384 well Corning plate (product #3765, Corning, NY) at 11,000 cells per well in a volume of 20 μL. The cells were dosed with the compounds of interest using an Echo Liquid Handler (Beckman Coulter, Brea, CA) at 50 μM top dose with 3-fold dilutions down to 0.002679 μM for a 10-point dose response. The plate was incubated for 3 hours at 37° C. and subsequently treated with recombinant mouse IFNγ (R&D Systems, Catalog #485-MI, Minneapolis, MN; 100 nM final concentration) for 10 minutes at 37° C. to induce STAT1 phosphorylation followed by 3.3 μM staurosporine treatment for 1 hour at 37° C. to terminate phosphorylation. Media was then aspirated and 20 μL of 1× lysis buffer/blocking reagent (Cisbio Phospho-STAT1 kit, part #63ADK026PEH, Bedford, MA) was added. The plate was placed on a plate shaker for 30 minutes at room temperature, sealed, and stored at −80° C. until needed. To thaw, the plate was placed on shaker at room temperature until completely thawed while the antibody master mix was made (1:40 Phospho-STAT1 Eu Cryptate antibody, 1:40 Phospho-STAT1 d2 antibody, with the detection buffer; Cisbio Phospho-STAT1 kit, part #63ADK026PEH, Bedford, MA). The antibody master mix was then dispensed at 4 μL per well into a 384 ProxiPlate Plus (PerkinElmer, part #6008289, Waltham, MA) and 16 μL of lysate was added from the Corning plate to the Proxiplate using a VIAFLO 384 (INTEGRA). The plate was incubated for 3 hours at room temperature and read on an EnVision® (Perkin Elmer) plate reader with laser excitation at 335 nm and emission at 665 nm. Dotmatics Studies (Bishop's Stortford, UK) was utilized to generate all dose-response curves and calculate EC50s and are shown in Table 2.

TABLE 2
EC₅₀ values in B16F10 IFNγ-induced STAT1 phosphorylation
(pSTAT1) cell assay.

| | B16F10 IFNγ Induced | | B16F10 IFNγ Induced |
|---|---|---|---|
| Compound | pSTAT1 | Compound | pSTAT1 |
| No. | EC₅₀ (μM) | No. | EC₅₀ (μM) |
| 100 | 0.164 | 103 | 0.528 |
| 104 | 3.35 | 105 | 4.81 |
| 106 | 11.9 | 107 | 3.96 |
| 108 | 1.76 | 109 | 6.03 |
| 110 | 6.99 | 111 | 7.98 |
| 112 | 1.21 | 113 | 2.03 |
| 114 | 1.78 | 115 | 1.83 |
| 116 | 17.2 | 117 | 0.723 |
| 118 | 4.22 | 119 | 6.66 |
| 120 | 1.01 | 121 | 8.15 |
| 122 | 6.39 | 123 | 2.76 |
| 124 | 6.94 | 125 | >50 |
| 126 | 25.5 | 127 | 8.97 |
| 128 | 25.7 | 129 | 0.589 |
| 130 | 4.11 | 131 | >50 |
| 132 | 1.58 | 133 | 0.762 |
| 134 | >50 | 135 | 0.688 |
| 136 | 27.6 | 137 | >50 |
| 138 | 1.48 | 139 | >50 |
| 140 | 2.95 | 141 | 6.97 |

### Example 46: Comparative Mobility Shift Assay (MSA) and B16F10 pSTAT1 HTRF Proximal Pharmacodynamic (PD) Assay for Thiochromane Compounds of the Disclosure and Corresponding Chromane Analogs

Assay data was obtained using the protocols described in Example 44 for PTPN2 mobility shift assay data (biochemical potency) and in Example 45 for B16F10 pSTAT1 proximal pharmacodynamic (PD) assay data (cellular potency). The data comparing thiochromane analogs of the disclosure with corresponding chromane analogs and are shown in Table 3. Eleven of the thirteen thiochromanes exceed the corresponding chromane analogs in biochemical potency as shown in the PTPN2 MSA. Nine of the eleven thiochromanes exceed the corresponding chromane analogs in cellular potency in the pSTAT1 proximal pharmacodynamic assay. For nine of the eleven comparisons, the thiochromane analogs showed greater potency enhancement in the cellular assay over the potency enhancement observed in the corresponding biochemical comparison.

TABLE 3
IC₅₀ values of exemplary thiochromane compounds of the disclosure in the
PTPN2 Mobility Shift Assays (MSA) and EC₅₀ values in B16F10 IFNγ-induced STAT1
phosphorylation (pSTAT1) cell assays compared with corresponding chromane analogs.

| 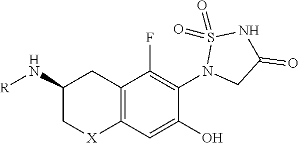 | | | | | | | | |
|---|---|---|---|---|---|---|---|---|
| | | PTPN2 | pSTAT | | PTPN2 | | | |
| | X = S | MSA | 1 | X = O | MSA | pSTAT1 | Thiochromane | Thiochromane |
| | Compound | IC₅₀ | EC₅₀ | Comparator | IC₅₀ | EC₅₀ | x-fold>potency | x-fold>potency |
| R— | # | (μM) | (μM) | # | (μM) | (μM) | biochemical | cellular |
| (F)₂HC— | 100 | 0.002 | | C-1 | 0.008 | 3.6 | 3.4 | |
| CH₂CH₂CH₂— | | 6 | | | 85 | | | |
| H— | 102 | 0.031 | | C-2 | 0.052 | 5.75 | 1.7 | |
| | | 4 | | | 9 | | | |
| (CH₃)₂HC—CH₂CH₂— | 103 | 0.001 | 0.528 | C-3 | 0.015 | 6.96 | 11.9 | 13.2 |
| | | 34 | | | 9 | | | |
| 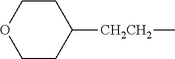 | 109 | 0.009 02 | 6.03 | C-4 | 0.018 1 | 32.2 | 2.0 | 5.3 |
| 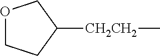 | 114 | 0.005 9 | 1.78 | C-5 | 0.035 6 | 22.6 | 6.0 | 12.7 |
| 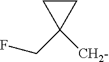 | 128 | 0.040 6 | 25.7 | C-6 | 0.049 5 | >50 | 1.2 | >1.9 |
| 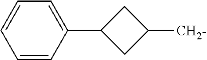 | 135 | 0.004 26 | 0.688 | C-7 | 0.008 28 | >50 | 1.9 | >72.7 |
| 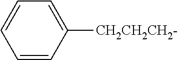 | 136 | 0.025 4 | 27.6 | C-8 | 0.019 1 | >50 | 0.8 | >1.8 |
| (F)₂HC—CH₂ O— | 137 | 0.021 | >50 | C-9 | 0.023 | >50 | 1.1 | ~1.0 |
| CH₂CH₂CH₂— | | | | | 9 | | | |
| 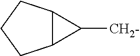 | 138 | 0.015 9 | 1.48 | C-10 | 0.032 4 | >50 | 2.0 | >33.8 |
| 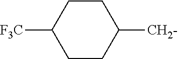 | 139 | 0.004 95 | >50 | C-11 | 0.026 6 | >50 | 5.4 | ~1.0 |
| 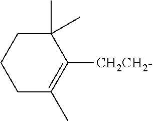 | 140 | 0.014 8 | 2.95 | C-12 | 0.022 6 | >50 | 1.5 | >16.9 |
| 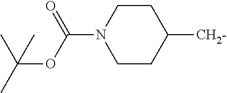 | 141 | 0.014 1 | 6.97 | C-13 | 0.014 8 | >50 | 1.0 | >7.2 |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound represented by Formula (I): 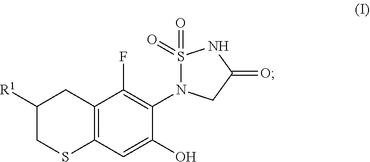
or a pharmaceutically acceptable salt thereof, wherein:
R¹ is selected from the group consisting of —NH₂, —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl;
wherein —N(R^{a})—C₁₋₈alkyl, —N(R^{a})—C₂₋₆alkenyl —N(R^{a})—C₁₋₆alkylene-C₃₋₆cycloalkyl, —N(R^{a})—C(O)—O—C₁₋₆alkyl, —N(R^{a})—C₁₋₆alkylene-4-7 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
wherein if —N(R)—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}; and
R^{g} is independently selected, for each occurrence, from the group consisting of halogen, hydroxyl, C₁₋₆alkyl, phenyl, and C₁₋₆alkoxy, wherein C₁₋₆alkyl, phenyl, or C₁₋₆alkoxy may optionally be substituted by one, two three or more substituents each independently selected from R^{P};
R^{h} is independently selected, for each occurrence, from the group consisting of C₁₋₆alkyl and C₁₋₆alkyl-O—C(O)—;
R^{P} is independently selected, for each occurrence, from the group consisting of C₁₋₆alkyl, halogen and hydroxyl; and
R^{a} is independently selected, for each occurrence, from the group consisting of hydrogen and C₁₋₆alkyl.

2. The compound of claim 1, wherein R¹ is —N(H)—C₁₋₈alkyl, wherein C₁₋₈alkyl is optionally substituted with R^{g}.

3. The compound of claim 2, wherein R^{g} is selected from fluoro, hydroxyl, C₁₋₆alkyl, and C₁₋₆alkoxy, wherein C₁₋₆alkyl, and C₁₋₆alkoxy are optionally substituted by one, two three or more substituents selected from R^{P}; and
R^{P} is independently, for each occurrence, from halogen.

4. The compound of either of claims 2 or 3, wherein R¹ is selected from the group consisting of

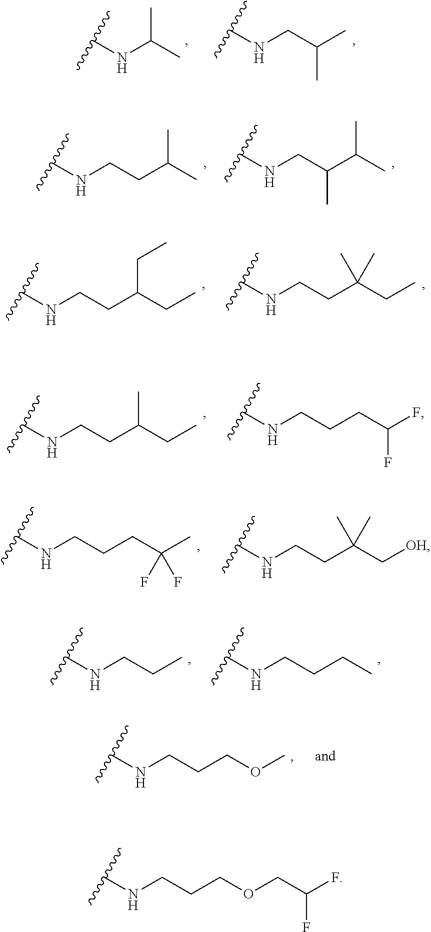

5. The compound of claim 1, wherein R¹ is —N(H)—C₁₋₆alkylene-C₃₋₆cycloalkyl, wherein —N(H)—C₁₋₆alkylene-C₃₋₆cycloalkyl may optionally be substituted by one, two, three or more substituents each independently selected from R^{g}.

6. The compound of claim 5, wherein R^{g} is independently selected, for each occurrence, from the group consisting of fluoro, C₁₋₆alkyl, and phenyl, wherein C₁₋₆alkyl and phenyl are optionally substituted by one, two three or more substituents selected from R^{P}; and
R^{P} is independently selected, for each occurrence, from fluoro or hydroxyl.

7. The compound of either of claims 5 or 6, wherein R¹ is selected from the group consisting of

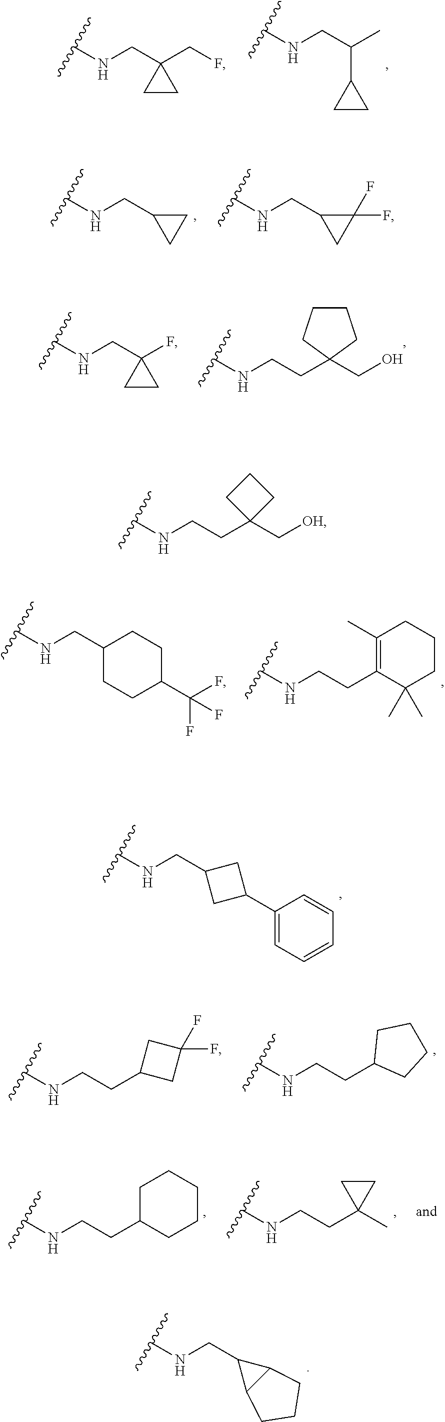

8. The compound of claim 1, wherein R¹ is —N(H)—C₁₋₆alkylene-4-7 membered heterocyclyl, wherein —N(H)—C₁₋₆alkylene-4-7 membered heterocyclyl may optionally be substituted by one, two, three or more substituents each independently selected from R^{g}, wherein if the 4-7 membered heterocyclyl contains a substitutable ring nitrogen atom, that ring nitrogen atom may optionally be substituted by R^{h}.

9. The compound of claim 8, wherein R^{h} is C₁₋₆alkyl-O—C(O)—.

10. The compound of claim 8 or 9, wherein R¹ is selected from the group consisting of

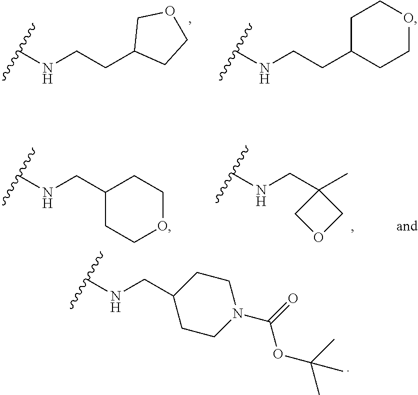

11. The compound of claim 1, wherein R¹ is —N(H)—C₁₋₆alkylene-phenyl, wherein —N(H)—C₁₋₆alkylene-phenyl may optionally be substituted on one or more available carbons by one, two, three or more substituents each independently selected from R^{g}.

12. The compound of claim 11, wherein R^{g} is independently, for each occurrence, C₁₋₆alkyl.

13. The compound of claim 11, wherein R¹ is selected from the group consisting of

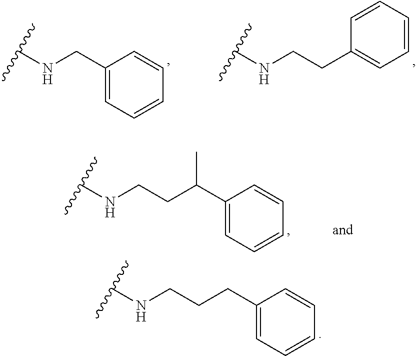

14. The compound of claim 1, wherein R¹ is —N(H)—C₁₋₆alkylene-5-6 membered heteroaryl, wherein —N(H)—C₁₋₆alkylene-5-6 membered heteroaryl may optionally be substituted on one or more available carbons by one, two, three or more substituents each independently selected from R^{g}.

15. The compound of claim 14, wherein R^{g} is independently, for each occurrence, C₁₋₆alkyl.

16. The compound of claim 14, wherein R¹ is

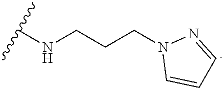 [SMILES: *NCCCn1cccn1]

17. A compound selected from the group consisting of
5-{(3S)-3-[(4,4-difluorobutyl)amino]-5-fluoro-7-hydroxy-3,4-dihydro-2H-1-benzothiopyran-6-yl}-1λ⁶,2,5-thiadiazolidine-1,1,3-trione;
tert-butyl [(3S)-5-fluoro-7-hydroxy-6-(1,1,4-trioxo-1λ⁶,2,5-thiadiazolidin-2-yl)-3,4-dihydro-2H-1-benzothiopyran-3-yl]carbamate;
5-[(3S)-3-amino-5-fluoro-7-hydroxy-3,4-dihydro-2H-1-benzothiopyran-6-yl]-1′⁶,2,5-thiadiazolidine-1,1,3-trione;
5-{(3S)-5-fluoro-7-hydroxy-3-[(3-methylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-({2-[1-(hydroxymethyl)cyclobutyl]ethyl}amino)-3,4-dihydro-2H-1-benzothiopyran-6-yl]-1′⁶,2,5-thiadiazolidine-1,1,3-trione;
5-[(3S)-5-fluoro-7-hydroxy-3-({2-[1-(hydroxymethyl)cyclopentyl]ethyl}amino)-3,4-dihydro-2H-1-benzothiopyran-6-yl]-1′⁶,2,5-thiadiazolidine-1,1,3-trione;
5-{(3S)-5-fluoro-7-hydroxy-3-[(4-hydroxy-3,3-dimethylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-(propylamino)-3,4-dihydro-2H-1-benzothiopyran-6-yl]-1λ⁶,2,5-thiadiazolidine-1,1,3-trione;
5-{(3S)-5-fluoro-7-hydroxy-3-[(3-methylpentyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[2-(oxan-4-yl)ethyl]amino}-3,4-dihydro-2H-1-benzothiopyran-6-yl]-1λ⁶,2,5-thiadiazolidine-1,1,3-trione;
5-[(3S)-5-fluoro-7-hydroxy-3-{[(oxan-4-yl)methyl]amino}-3,4-dihydro-2H-1-benzothiopyran-6-yl]-1λ⁶,2,5-thiadiazolidine-1,1,3-trione;
5-{(3S)-3-[(cyclopropylmethyl)amino]-5-fluoro-7-hydroxy-3,4-dihydro-2H-1-benzothiopyran-6-yl}-1λ⁶,2,5-thiadiazolidine-1,1,3-trione;
5-[(3S)-3-(butylamino)-5-fluoro-7-hydroxy-3,4-dihydro-2H-1-benzothiopyran-6-yl]-1λ⁶,2,5-thiadiazolidine-1,1,3-trione;
5-{(3S)-5-fluoro-7-hydroxy-3-[(2-methylpropyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[2-(oxolan-3-yl)ethyl]amino}-3,4-dihydro-2H-1-benzothiopyran-6-yl]-1,2,5-thiadiazolidine-1,1,3-trione;
5-{(3S)-3-[(2,3-dimethylbutyl)amino]-5-fluoro-7-hydroxy-3,4-dihydro-2H-1-benzothiopyran-6-yl}-1λ⁶,2,5-thiadiazolidine-1,1,3-trione;
5-{(3S)-5-fluoro-7-hydroxy-3-[(propan-2-yl)amino]-3,4-dihydro-2H-1-benzothiopyran-6-yl}-1λ⁶,2,5-thiadiazolidine-1,1,3-trione;
5-[(3S)-3-[2-(3,3-difluorocyclobutyl)ethyl]amino}-5-fluoro-7-hydroxy-3,4-dihydro-2H-1-benzothiopyran-6-yl]-1λ⁶,2,5-thiadiazolidine-1,1,3-trione;
5-{(3S)-3-[(2-cyclohexylethyl)amino]-5-fluoro-7-hydroxy-3,4-dihydro-2H-1-benzothiopyran-6-yl}-1λ⁶,2,5-thiadiazolidine-1,1,3-trione;
5-{(3S)-3-[(3-ethylpentyl)amino]-5-fluoro-7-hydroxy-3,4-dihydro-2H-1-benzothiopyran-6-yl}-1,2,5-thiadiazolidine-1,1,3-trione;
5-{(3S)-3-[(2-cyclopentylethyl)amino]-5-fluoro-7-hydroxy-3,4-dihydro-2H-1-benzothiopyran-6-yl}-1λ⁶,2,5-thiadiazolidine-1,1,3-trione;
5-[(3S)-3-(benzylamino)-5-fluoro-7-hydroxy-3,4-dihydro-2H-1-benzothiopyran-6-yl]-1λ⁶,2,5-thiadiazolidine-1,1,3-trione;
5-{(3S)-5-fluoro-7-hydroxy-3-[(3-methylbut-2-en-1-yl)amino]-3,4-dihydro-2H-1-benzothiopyran-6-yl}-1λ⁶,2,5-thiadiazolidine-1,1,3-trione;
5-{(3S)-5-fluoro-7-hydroxy-3-[(2-phenylethyl)amino]-3,4-dihydro-2H-1-benzothiopyran-6-yl}-1λ⁶,2,5-thiadiazolidine-1,1,3-trione;
5-{(3S)-3-[(3,3-dimethylpentyl)amino]-5-fluoro-7-hydroxy-3,4-dihydro-2H-1-benzothiopyran-6-yl}-1λ⁶,2,5-thiadiazolidine-1,1,3-trione;
5-[(3S)-5-fluoro-3-1{[(1-fluorocyclopropyl)methyl]amino}-7-hydroxy-3,4-dihydro-2H-1-benzothiopyran-6-yl]-1λ⁶,2,5-thiadiazolidine-1,1,3-trione;
5-[(3S)-3-{[(2,2-difluorocyclopropyl)methyl]amino}-5-fluoro-7-hydroxy-3,4-dihydro-2H-1-benzothiopyran-6-yl]-1λ⁶,2,5-thiadiazolidine-1,1,3-trione;
5-[(3S)-5-fluoro-7-hydroxy-3-1{[(3-methyloxetan-3-yl)methyl]amino}-3,4-dihydro-2H-1-benzothiopyran-6-yl]-1λ⁶,2,5-thiadiazolidine-1,1,3-trione;
5-[(3S)-5-fluoro-3-({[1-(fluoromethyl)cyclopropyl]methyl}amino)-7-hydroxy-3,4-dihydro-2H-1-benzothiopyran-6-yl]-1λ⁶,2,5-thiadiazolidine-1,1,3-trione;
5-{(3S)-3-[(4,4-difluoropentyl)amino]-5-fluoro-7-hydroxy-3,4-dihydro-2H-1-benzothiopyran-6-yl}-1λ⁶,2,5-thiadiazolidine-1,1,3-trione;
5-{(3S)-5-fluoro-7-hydroxy-3-[(3-methoxypropyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[3-(1H-pyrazol-1-yl)propyl]amino}-3,4-dihydro-2H-1-benzothiopyran-6-yl]-1λ⁶,2,5-thiadiazolidine-1,1,3-trione;
5-[(3S)-5-fluoro-7-hydroxy-3-{[2-(1-methylcyclopropyl)ethyl]amino}-3,4-dihydro-2H-1-benzothiopyran-6-yl]-1λ⁶,2,5-thiadiazolidine-1,1,3-trione;
5-{(3S)-3-[(2-cyclopropylpropyl)amino]-5-fluoro-7-hydroxy-3,4-dihydro-2H-1-benzothiopyran-6-yl}-1λ⁶,2,5-thiadiazolidine-1,1,3-trione;
5-{(3S)-5-fluoro-7-hydroxy-3-[(3-phenylbutyl)amino]-3,4-dihydro-2H-1-benzothiopyran-6-yl}-1λ⁶,2,5-thiadiazolidine-1,1,3-trione;
5-[(3S)-5-fluoro-7-hydroxy-3-{[(3-phenylcyclobutyl)methyl]amino}-3,4-dihydro-2H-1-benzothiopyran-6-yl]-1λ⁶,2,5-thiadiazolidine-1,1,3-trione;
5-{(3S)-5-fluoro-7-hydroxy-3-[(3-phenylpropyl)amino]-3,4-dihydro-2H-1-benzothiopyran-6-yl}-1λ⁶,2,5-thiadiazolidine-1,1,3-trione;
5-[(3S)-3-{[3-(2,2-difluoroethoxy)propyl]amino}-5-fluoro-7-hydroxy-3,4-dihydro-2H-1-benzothiopyran-6-yl]-1λ⁶,2,5-thiadiazolidine-1,1,3-trione;
5-[(3S)-3-({[(1RS,5SR)-bicyclo[3.1.0]hexan-6-yl]methyl}amino)-5-fluoro-7-hydroxy-3,4-dihydro-2H-1-benzothiopyran-6-yl]-1λ⁶,2,5-thiadiazolidine-1,1,3-trione;
5-[(3S)-5-fluoro-7-hydroxy-3-({[4-(trifluoromethyl)cyclohexyl]methyl}amino)-3,4-dihydro-2H-1-benzothiopyran-6-yl]-1λ⁶,2,5-thiadiazolidine-1,1,3-trione;
5-[(3S)-5-fluoro-7-hydroxy-3-{[2-(2,6,6-trimethylcyclohex-1-en-1-yl)ethyl]amino}-3,4-dihydro-2H-1-benzothiopyran-6-yl]-1λ⁶,2,5-thiadiazolidine-1,1,3-trione; and
tert-butyl 4-({[(3S)-5-fluoro-7-hydroxy-6-(1,1,4-trioxo-1λ⁶,2,5-thiadiazolidin-2-yl)-3,4-dihydro-2H-1-benzothiopyran-3-yl]amino}methyl)piperidine-1-carboxylate,
or a pharmaceutically acceptable salt thereof.

18. A pharmaceutically acceptable composition comprising a compound of any one of claims 1-17 and a pharmaceutically acceptable carrier.
